(19) United States
(12) Patent Application Publication
(10) Pub. No.: US 2013/0040049 A1
(43) Pub. Date: Feb. 14, 2013

(54) "Growth from surface" Methodology for the Fabrication of Functional Dual Phase Conducting Polymer polypyrrole/polycarbazole/Polythiophene (CP/polyPyr/polyCbz/PolyTh)-Carbon Nanotube (CNT) Composites of Controlled Morphology and Composition - Sidewall versus End-Selective PolyTh Deposition

(71) Applicants: Jean-Paul Lellouche, Ashdod (IL); Diana Goldman, Nahariya (IL)

(72) Inventors: Jean-Paul Lellouche, Ashdod (IL); Diana Goldman, Nahariya (IL)

(73) Assignee: BAR-ILAN UNIVERSITY, Ramat Gan (IL)

(21) Appl. No.: 13/642,961

(22) PCT Filed: Apr. 14, 2011
(86) PCT No.: PCT/IL11/00317

§ 371 Date: Oct. 31, 2012

Related U.S. Application Data
(60) Provisional application No. 61/282,935, filed on Apr. 26, 2010.
(60) Provisional application No. 61/457,435, filed on Mar. 28, 2011.

(51) Int. Cl. C07D 333/02; B05D 5/12; B82Y 30/00; B82Y 40/00

(52) U.S. Cl. 427/113; 549/79; 977/746; 977/847

(57) ABSTRACT

A “growth from the surface” method for selectively depositing oxidative Liquid Phase Polymerizations (LPPs) onto the carbon nanotube (CNT) surface, said method comprising steps of: a. obtaining Multi-walled Carbon Nanotubes (MWC-NT); b. oxidized said MWCNTs to obtain oxidized COOH-MWCNTs; thereby (a) carboxylative opening oxidation-sensitive end-caps (polyCOOH end cluster); and, (b) introducing defect carboxylic (COOH) groups onto predetermined areas of said oxidized COOH-MWCNTs; c. COOH activating the polyCOOH shell using various COOH activating species; and, d. executing Liquid Phase Polymerization (LPP) oxidative depositing polymers selected from said polyCOOH polyTh-CP polymers, polyCOOH poly-Th-, polyEDOT (PEDOT)-, polyTh polyCOOH poly(thiophenyl-3 acetic acid, thiophenyl-3 acetic acid/EDOT, polyX, wherein X is elected from COOH, OH, NH2, polyCbz/polyPyr CP polymers and related combinatorial mixtures, polyCOOH PEDOT-poly(thiophenyl-3 acetic acid)′ thereby selectively depositing said oxidative LPPs onto said CNT surface.

### FIELD OF THE INVENTION

The present invention relates generally to Novel “Growth from” Methodology for the Fabrication of Functional Dual Phase Conducting Polymer polypyrrole/polycarbazole/Polythiophene (CP/polyPyr/polyCbz/PolyTh)-Carbon Nanotube (CNT) Composites of Controlled Morphology and Composition—Sidewall versus End-Selective PolyTh Deposition.

### BACKGROUND OF THE INVENTION

Since their discovery in 1991 (see Iijima, S, Nature 1991, 354, 56) as new nanosized materials, carbon nanotubes (CNTs) have been used in many research areas and applications ranging from nano-electronics to biomedical devices (see Dresselhaus, M. S.; Dresselhaus, G.; Eklund, P. Science of Fullerenes and Carbon Nanotubes; Academic Press: New York, 1996; Ajayan, P. M. Chem. Rev. 1999, 99, 1787; Poncharal, P.; Wang, Z. L.; Ugarte, D.; de Heer, W. A. Science 1999, 283, 1513). Biphasic composite materials based on the assembling of both conducting polymer (CP) and CNT phases often demonstrated combined and synergistic properties arising from each individual interacting component.5 Incorporating CNTs into CP matrices offers an attractive route to mechanically reinforce the polymer phase as well as to engineer novel electronic properties based on morphological modifications and/or electronic interactions between both components within composite phases. Generally speaking, three main approaches have been used to prepare such biphasic CP/CNT composites: (i) the direct mixing of pre-formed CPs with CNTs, (ii) the oxidative Liquid Phase chemical Polymerization (LPP) of CP-monomers/precursors in the presence of CNTs, and finally (iii) the electrochemical oxidation of same CP-monomers/precursors onto electrodes in the presence of CNTs. In this context, few leading examples included electrochemically deposited polyaniline (PANT) films onto CNT-modified electrodes (sensing technology), CNT-loaded poly(p-phenylene vinylene) (PPV) composites12 (fabrication of LED thin-films applied to molecular opto-electronics), and poly(mphenylene vinylene-co-2,5-dioctoxy-p-phenylene) (PmPV)-SWCNT composites (self-assembled electronically tunable nanoscale sensors). TEM and SEM microscopies invariably showed embedded CNTs in different states of exfoliation and/or aggregation (see Downs, C.; Nuget, J.; Ajayan, P. M.; Duquette, D. J.; Santhanam, K. S. V. Adv. Mater. 1999, 12, 1028; Valter, B.; Ram, M. K.; Nicolini, C. Langmuir 2002, 18, 1535; Curran, S. A.; Ajayan, P. M.; Blau, W. J.; Carroll, D. L.; Coleman, J. N.; Dalton, A. B.; Davey, A. P.; Drury, A.; McCarthy, B.; Maier, S.; Strevens, A. Adv. Mater. 1998, 10, 1091; Coleman, J. N.; Curran, S.; Dalton, A. B.; Davey, A. P.; McCarthy, B.; Blau, W.; Barklie, R. C. Phys. Rev. B 1998, 58, 7492; Star, A.; Stoddart, J. F.; Steuerman, D.; Diehl, M.; Boukai, A.; Wong, E. W.; Chung, S. W.; Choi, H.; Heath, J. R.; Angew. Chem. Int. Ed 2001, 40, 1721; Panhuis, M.; Maiti, A.; Dalton, A. B.; van den Noort, A.; Coleman, J. N.; McCarthy, B.; Blau, W. J.; J. Phys. Chem. B 2003, 107, 478; Dalton, A. B.; Byrne, H. J.; Coleman, J. N.; Curran, S.; Davey, A. P.; McCarthy, B.; Blau, W. Synth. Met. 1999, 102, 1176) while being held in corresponding CP polymeric matrices through weak π-πstacking and/or Van der Waals interactions between polymer backbones and CNT lattices.

The above-cited fabrication methodologies often underlooked one main critical issue that must be addressed for the obtainment of optimally tailored CP/CNT biphasic composites, i.e. the putative phase incompatibility that might develop during the dual phase rectional mixing. This may detrimentally result in composite materials that will disclose discrete non-interacting CNT and CP phases (phase heterogeneity). Even in extreme cases, one simply isolated non-modified starting CNTs. Another clear limitating issue concerned the fact that known CP/CNT biphasic composites rather incorporated heterocyclic non-functional CPs such as polypyrrole (polyPyr), polythiophene (polyTh), and poly(3,4-ethylenedioxy-thiophene) (polyPEDOT). This lack of monomer chemical functionality raises a legitimate concern about the generality of all the above-cited composite fabrication methods when dealing with functional CP-monomers/precursors.

Indeed, various oxidative LPP experiments that involved functional monocarboxylated Pyr- and carbazolyl (Cbz)-based monomers:

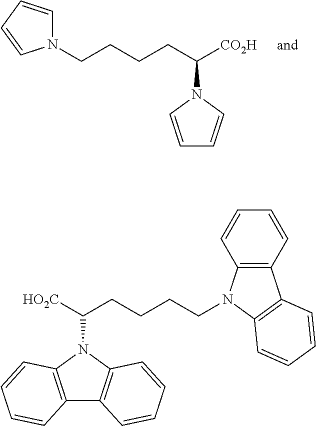

and CVD produced MWCNTs (MER Corporation Ltd) were recently conducted, but never afforded the corresponding expected biphasic CP/CNT composites. They rather left starting MWCNTs unchanged while LPP bulk polyPyr-poly(4a, see scheme 1) and polyCbz-poly(2b, see scheme 1) doped polymers having been produced and eliminated during composite purification.

Therefore, there is still a long felt need for a new methodological concept to overcome the above mentioned drawbacks.

### SUMMARY OF THE INVENTION

The present invention provides a new methodological concept so as to overcome the eliminatation of LPP bulk polyPyr-poly(4a, see scheme 1) and polyCbz-poly(2b, see scheme 1) doped polymers during composite purification.

And thus, providing a general solution that will address the inherent phase compatibility issue cited above.

The core concept behind the method of the present invention 3 main steps:
(a) the Carbon Nanotube (CNT) oxidation;
(b) the linker (Pyr/Cbz and Th-based ones) covalent attachment;
(c) the polymer growth from the surface using the oxidative LPP protocol for growing conducting polymers of all the series of polypyrrole, polycarbazole and polythiophene.

The innovative concept of the present invention combined two key steps, i.e. (i) first, covalently coupling/grafting oxidized polycarboxylated MWCNTs (c-MWCNTs) with hydroxylated or aminated polypyrrole/carbazolyl (Pyr/Cbz)-containing linkers 1a-b and 2a (scheme. 1, bottom left) affording Pyr/Cbz-decorated d-MWCNTs; and (ii), second, in-situ oxidatively polymerizing various functional monocarboxylated Pyr/Cbzmonomers 3-4a and 2b (LPP conditions) in the presence of former d-MWCNTs, as further described in the following scheme:

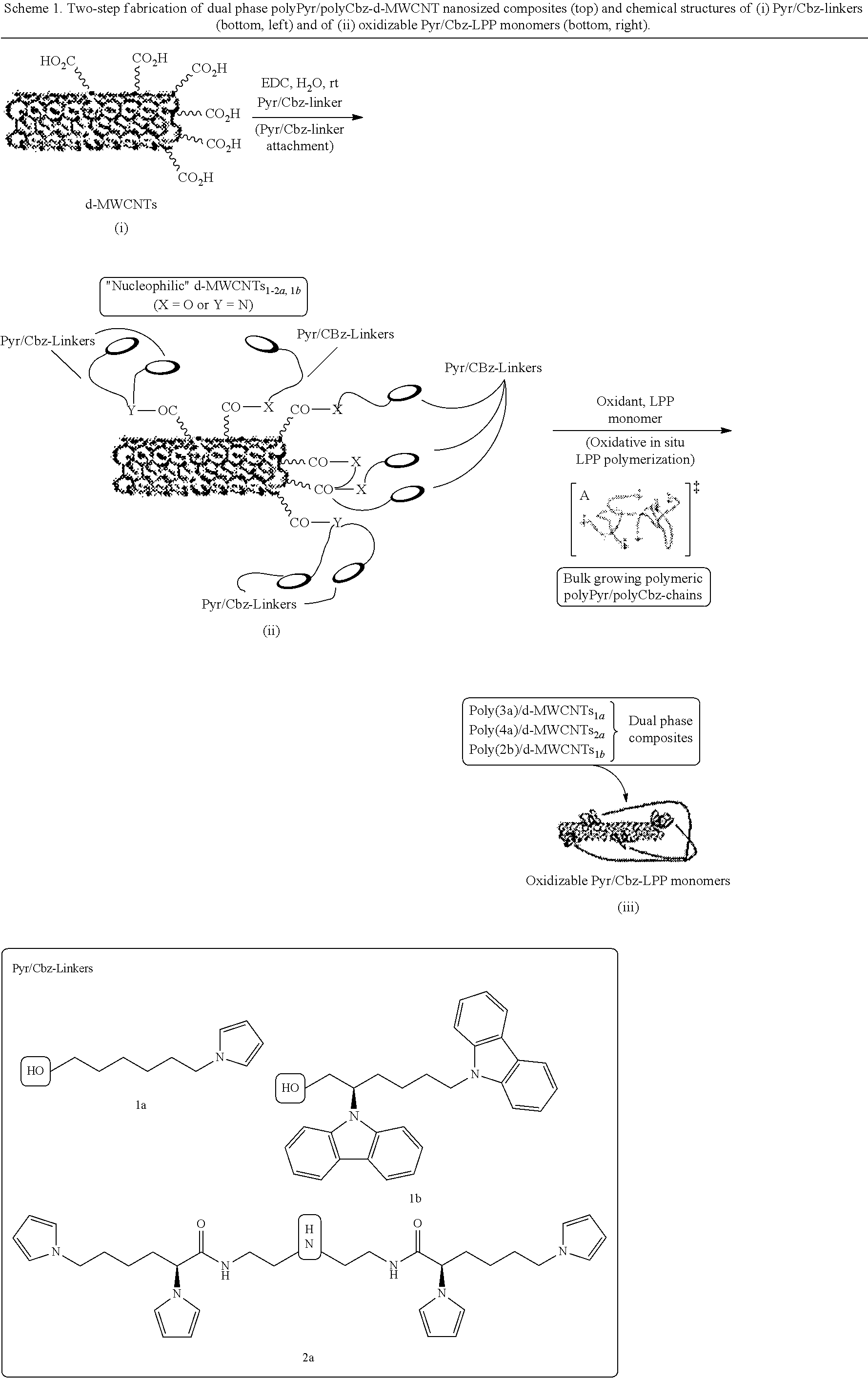

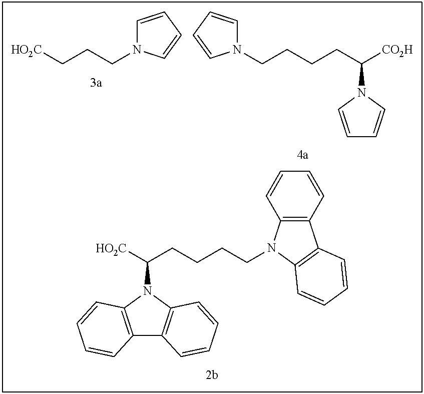

As a matter of consequence, this intermediate d-MWCNT1-2a,1b phase will now compete with bulk Pyr/Cbz-monomers for CP chain elongation.

It will act as a “nucleophilic” nanosized phase able to trap the cation/radical electrophilic polyPyr/polyCbz-CP polymer chains of type A (see scheme 1) generated in the bulk medium during LPP experiments.

In this way, corresponding functional polycarboxylated (polyCOOH) polyPyr/polyCbz-polymers will be grown and deposited onto d-MWCNTs1-2a,1b leading to novel CP/CNT biphasic composite materials poly(3-4a,2b)/d-MWCNTs1-2a,1b. In addition, underlayer weight depositions of CP polymers may be strictly controlled using unique sets of LPP rectional parameters optimized by statistically relevant Design Of Experiments (DOE). In order to demonstrate this, the illustrative case of the DOE-optimized preparation of the poly(4a)/d-MWCNTs2a composite has been reported below as part of these studies. Consequently, morphologically versatile CP/CNT composite materials that contained controlled weight ratios of both CP/CNT phases have been readily fabricated applying this LPP methodological variant.

Thus, the present invention discloses and provides:

(a) a novel LPP set of conditions involving gthe use of “nucleophilized” Th-decorated oxidized MWCNTs for the controlled growth/deposition of polyCOOH polyTh-, polyEDOT (PEDOT)-, and combinatorial mixtures [polyCOOH PEDOT-poly(thiophenyl-3 acetic acid)] phase onto specific areas, i.e., sidewalls and CNT extremities or topologically selectively at only oxidized extremities of pegylated oxidized polyTh-decorated MWCNTs.
(b) examples for the deposition of a polyTh polyCOOH poly(thiophenyl-3 acetic acid) phase but the process has been extended to EDOT (PEDOT polyTh precursor) and/or to combinatorial mixtures of thiophenyl-3 acetic acid/EDOT. A similar strategy has been extended to similar combinatorial mixtures of functional polyX (X: COOH, OH, NH₂) polyCbz/polyPyr CO polymers.
(c) full characterization proving the topologically selective deposition of the LPP polyTh phase has been provided emphasizing the exact morphologies of resulting composites. Similar characterization works have been described in an article published by Diana Goldman and J.-P. Lellouche, An easy method for the production of functional polypyrrole/MWCNT and polycarbazole/MWCNT composites using nucleophilic multi-walled carbon nanotubes, Carbon, 2010, 48, 4170-4177.
(d) various passivating hydrophobic/amphiphilic polymers have been also used fullfilling the same COOH protective role than the PEG polymers used in the studies mentioned above, for example polyvinylpyrrolidone (PVP), polycarbonates (PCs), polyesters (PEs) and polysiloxanes.

It is one object of the present invention to provide a “growth from surface” method for selectively depositing oxidative Liquid Phase Polymerizations (LPPs) onto the carbon nanotubes (CNT) surface, said method comprising steps of:
a. obtaining Multi-walled Carbon Nanotubes (MWCNT);
b. oxidized said MWCNTs to obtain oxidized COOH-MWCNTs; thereby (a) carboxylative opening oxidation-sensitive end-caps (polyCOOH end cluster); and, (b) introducing defect carboxylic (COOH) groups onto predetermined areas of said oxidized COOH-MWCNTs;
c. COOH activating the polyCOOH shell (namely, the surface) using various COOH activating species; and, [it is emphasized that in this step, as will be described herein after, the linkers (e.g. Pyr/Cbz and Th-based ones) are covalent attached to the oxidized COOH-MWCNTs];
d. executing Liquid Phase Polymerization (LPP) oxidative depositing polymers selected from said polyCOOH polyTh-CP polymers, polyCOOH polyTh-, polyEDOT (PEDOT)-, polyTh polyCOOH poly(thiophenyl-3 acetic acid, thiophenyl-3 acetic acid/EDOT, polyX, wherein X is elected from COOH, OH, NH₂, polyCbz/polyPyr, CP polymers and related combinatorial mixtures, polyCOOH PEDOT-poly(thiophenyl-3 acetic acid)′ thereby selectively depositing said oxidative LPPs onto said CNT surface.

It is another object of the present invention to provide the method as defined above, wherein said selectively deposition is performed in a controlled manner and for controlled polymer deposited amounts.

It is another object of the present invention to provide the method as defined above, wherein said predetermined area are selected from a group consisting of sidewall surfaces of said oxidized COOH-MWCNTs or CNT extremities or topologically selectively at only oxidized extremities of pegylated oxidized polyTh-decorated MWCNTs, end-decorated, selectively end-decorated Th-CNTs and any combination thereof.

It is another object of the present invention to provide the method as defined above, wherein said step of obtaining Multi-walled Carbon Nanotubes (MWCNT) is performed by chemical vapor deposition (CVD) and possess average diameters/lengths of 140±30 nm/7±2 nm respectively.

It is another object of the present invention to provide the method as defined above, wherein said MWCNT are composed of about 340 to about 530 graphitic layers and disclose purity higher than 90% as determined by thermogravimetric analysis (TGA)]; It is another object of the present invention to provide the method as defined above, wherein said step of oxidizing said MWCNT is performed by known wet-chemistry protocol.

It is another object of the present invention to provide the method as defined above, wherein said step of oxidizing said MWCNT by known wet-chemistry protocol is performed by steps of (a) oxidative acidic 1/1 v/v mixture of concentrated 12M HNO₃ and 36M H₂SO₄ (70° C., 2 h); (b) multiple rinsing with bi-distilled H₂O until neutrality.

It is another object of the present invention to provide the method as defined above, wherein said steps of (a) carboxylative opening oxidation-sensitive end-caps, namely, polyCOOH end cluster; and, (b) introducing defect carboxylic (COOH) groups on sidewall surfaces of said oxidized COOH-MWCNTs; are performed simultaneously. It is another object of the present invention to provide the method as defined above, wherein said step of COOH activating the polyCOOH shell is performed by steps of (a) admixing aqueous N′-(3-dimethylaminopropyl)-N-ethyl-carbodiimide (EDC); (b) covalently attaching at least one selected from a group consisting of Thp-containing linker, thiophene-3-ethanol, hydroxylated or aminated polypyrrole/carbazolyl (Pyr/Cbz)-containing linkers; Pyr/Cbz/Th bulk monomers; Pyr/Cbz/Th linkers and any combination thereof.

It is another object of the present invention to provide the method as defined above, wherein said step of COOH activating the polyCOOH shell is performed by using about 3.0 mg or about 15 mmoles of EDC.

It is another object of the present invention to provide the method as defined above, wherein said step of COOH activating the polyCOOH shell is performed by using about 1.0 mL H₂O.

It is another object of the present invention to provide the method as defined above, wherein said step of COOH activating the polyCOOH shell is performed for about 1 h. It is another object of the present invention to provide the method as defined above, wherein said step of COOH activating the polyCOOH shell is performed at room temperature.

It is another object of the present invention to provide the method as defined above, wherein said step of covalently attaching at least one selected from a group consisting of Thp-containing linker, thiophene-3-ethanol, hydroxylated or aminated polypyrrole/carbazolyl (Pyr/Cbz)-containing linkers; Pyr/Cbz/Th bulk monomers; Pyr/Cbz/Th linkers and any combination thereof is performed by adding said linker 1.0 equiv./EDC in about 1.0 mL CH₃CN.

It is another object of the present invention to provide the method as defined above, wherein said step of covalently attaching is performed for about 10 hours.

It is another object of the present invention to provide the method as defined above, wherein said step of covalently attaching is performed at about room temperature.

It is another object of the present invention to provide the method as defined above, wherein said EDC reacts with MWCNT carboxylic acid groups to form an active O-acylisourea intermediate.

It is another object of the present invention to provide the method as defined above, wherein said intermediate can be easily displaced by nucleophilic attack using the corresponding hydroxylated Th-containing linker.

It is another object of the present invention to provide the method as defined above, wherein said step of COOH activating the polyCOOH shell is performed by using at least one selected from a group consisting of PEG-passivated oxidized MWCNTs, polyvinylpyrrolidone (PVP), polycarbonates (PCs), polyesters (PEs), polysiloxanes; and any combination thereof.

It is another object of the present invention to provide the method as defined above, wherein said PEG is cc, w-bis-methoxy PEG polymer.

It is another object of the present invention to provide the method as defined above, wherein the molecular weight of said PEG is MW=2,000 Daltons. It is another object of the present invention to provide the method as defined above, wherein about 30.0 mL to about 3.0 mL of distilled water of said PEG is used.

It is another object of the present invention to provide the method as defined above, wherein said step of PEG-passivated oxidized MWCNTs is performed for about 20 min incubation.

It is another object of the present invention to provide the method as defined above, wherein said step of PEG-passivated oxidized MWCNTs is performed at about 20° C.

It is another object of the present invention to provide the method as defined above, wherein said step of Liquid Phase Polymerization (LPP) oxidative deposing said polyCOOH polyTh-CP polymers is performed by at least one selected from a group consisting of (a) Th-containing MWCNT; (b) acidic Th-based LPP monomer thiophene-3-yl acetic acid.

It is another object of the present invention to provide the method as defined above, wherein said step of Liquid Phase Polymerization (LPP) oxidative deposing said polymers is performed by at least one selected from a group consisting of (i) former type of “nucleophilized” Th-containing MWCNTs; (ii) acidic Th-based LPP monomer thiophene-3-yl acetic acid; (iii) Pyr/polyPyr; (iv) Pyr/polyPyr; and any combination thereof.

It is another object of the present invention to provide the method as defined above, wherein said step of Liquid Phase Polymerization (LPP) oxidative deposing said polymers is performed while using cationic cetyltrimethylammonium bromide (CTAB) concentration in the rang of about 0.01 to about 0.1 M for at least 1 hour. It is another object of the present invention to provide the method as defined above, wherein said selectively deposition is performed in a Liquid Phase Polymerization conditions (LPP conditions) selected from a group consisting of (a) concentration of cationic cetyltrimethylammonium bromide surfactant (CTAB) in the range of about 0[M] to about 0.1M; (b) the amount of Thiophene-3-yl acetic acid is in the range of 10.0 mg to about 35 mg; (c) the amount of Thiophene-3-yl acetic acid is in the range of 0.01 mmol to about 0.2 mmol; (d) the amount of Oxidant is in the range of 1.0 equiv./Th-monomer to about 3.5 equiv./Th-monomer; (e) the amount of Monomer solvent is in the range of 1.0 mL to about 3.5 mL; (f) Temp. of polymerization is in the range of 0 degrees to about 10 degree; (g) Time of polymerization is in the range of 0.5 hours to about 2 hours; and any combination thereof.

It is another object of the present invention to provide the method as defined above, wherein said Oxidant is Anhydrous FeCl₃.

It is another object of the present invention to provide the method as defined above, wherein said Monomer solvent is Distilled CHCl₃.

It is another object of the present invention to provide a “growth from surface” method for fabricating functional dual phase Conducting Polymer/Polythiophene (CP/PolyTh)-Carbon Nanotube (CNT), comprising:
a. obtaining Multi-walled Carbon Nanotubes (MWCNTs);
b. oxidized said MWCNTs to obtain oxidized COOH-MWCNTs; thereby (a) carboxylative opening oxidation-sensitive end-caps (polyCOOH end cluster); and, (b) introducing defect carboxylic (COOH) groups onto predetermined areas of said oxidized COOH-MWCNTs;
c. COOH activating the polyCOOH shell (namely, the surface) using various COOH activating species;
d. Liquid Phase Polymerization (LPP) oxidative deposing polymers selected from said polyCOOH polyTh-CP polymers, polyCOOH polyTh-, polyEDOT (PEDOT)-, polyTh polyCOOH poly(thiophenyl-3 acetic acid, thiophenyl-3 acetic acid/EDOT, polyX, wherein X is elected from COOH, OH, NH₂, polyCbz/polyPyr CP polymers and related combinatorial mixtures, polyCOOH PEDOT-poly(thiophenyl-3 acetic acid); thereby providing said dual phase Conducting Polymer/Polythiophene (CP/PolyTh)-Carbon Nanotube (CNT).

It is another object of the present invention to provide the method as defined above, wherein said predetermined area are selected from a group consisting of sidewall surfaces of said oxidized COOH-MWCNTs or CNT extremities or topologically selectively at only oxidized extremities of pegylated oxidized polyTh-decorated MWCNTs, end-decorated, selectively end-decorated Th-CNTs and any combination thereof.

It is another object of the present invention to provide the method as defined above, wherein said step of obtaining Multi-walled Carbon Nanotubes (MWCNT) is obtained by chemical vapor deposition (CVD) and possess average diameters/lengths of 140±30 nm/7±2 nm respectively.

It is another object of the present invention to provide the method as defined above, wherein said MWCNT are composed of 340-530 graphitic layers and disclose purity higher than 90% as determined by thermogravimetric analysis (TGA).

It is another object of the present invention to provide the method as defined above, wherein said step of oxidizing said MWCNT is performed by known wet-chemistry protocol.

It is another object of the present invention to provide the method as defined above, wherein said step of oxidizing said MWCNT by known wet-chemistry protocol is performed by steps of (a) oxidative acidic 1/1 v/v mixture of concentrated 12M HNO₃ and 36M H₂SO₄ (70° C., 2 h); (b) multiple rinsing with bi-distilled H₂O until neutrality.

It is another object of the present invention to provide the method as defined above, wherein said steps of (a) carboxylative opening oxidation-sensitive end-caps (polyCOOH end cluster); and, (b) introducing defect carboxylic (COOH) groups on sidewall surfaces of said oxidized COOH-MWCNTs; are performed simultaneously.

It is another object of the present invention to provide the method as defined above, wherein said step of COOH activating the polyCOOH shell is performed by steps of (a) admixing aqueous N′-(3-dimethylaminopropyl)-N-ethyl-carbodiimide (EDC); (b) covalently attachhig at least one selected from a group consisting of Thp-containing linker, thiophene-3-ethanol, hydroxylated or aminated polypyrrole/carbazolyl (Pyr/Cbz)-containing linkers; Pyr/Cbz/Th bulk monomers; Pyr/Cbz/Th linkers and any combination thereof.

It is another object of the present invention to provide the method as defined above, wherein said step of COOH activating the polyCOOH shell is performed by using about 3.0 mg or about 15.7 mmoles of EDC.

It is another object of the present invention to provide the method as defined above, wherein said step of COOH activating the polyCOOH shell is performed for about 1 h.

It is another object of the present invention to provide the method as defined above, wherein said step of COOH activating the polyCOOH shell is performed at room temperature.

It is another object of the present invention to provide the method as defined above, wherein said step of covalently attaching at least one selected from a group consisting of Thp-containing linker, thiophene-3-ethanol, hydroxylated or aminated polypyrrole/carbazolyl (Pyr/Cbz)-containing linkers; Pyr/Cbz/Th bulk monomers; Pyr/Cbz/Th linkers and any combination thereof is performed by adding said linker 1.0 equiv./EDC in about 1.0 mL CH₃CN.

It is another object of the present invention to provide the method as defined above, wherein said step of covalently attaching is performed for about 10 hours.

It is another object of the present invention to provide the method as defined above, wherein said step of covalently attaching is performed at about room temperature.

It is another object of the present invention to provide the method as defined above, wherein said EDC reacts with MWCNT carboxylic acid groups to form an active O-acylisourea intermediate.

It is another object of the present invention to provide the method as defined above, wherein said intermediate can be easily displaced by nucleophilic attack using the corresponding hydroxylated Th-containing linker.

It is another object of the present invention to provide the method as defined above, wherein said step of COOH activating the polyCOOH shell is performed by using at least one selected from a group consisting of PEG-passivated oxidized MWCNTs, polyvinylpyrrolidone (PVP), polycarbonates (PCs), polyesters (PEs), polysiloxanes; and any combination thereof.

It is another object of the present invention to provide the method as defined above, wherein said PEG is a, co-bis-methoxy PEGpolymer.

It is another object of the present invention to provide the method as defined above,

wherein the molecular weight of said PEG is MW=2,000 Daltons.

It is another object of the present invention to provide the method as defined above, wherein about 30.0 mL to about 3.0 mL of distilled water of said PEG is used.

It is another object of the present invention to provide the method as defined above, wherein said step of PEG-passivated oxidized MWCNTs is performed for about 20 min incubation.

It is another object of the present invention to provide the method as defined above, wherein said step of PEG-passivated oxidized MWCNTs is performed at about 20° C.

It is another object of the present invention to provide the method as defined above, wherein said step of Liquid Phase Polymerization (LPP) oxidative deposing said polyCOOH polyTh-CP polymers is performed by at least one selected from a group consisting of (a) Th-containing MWCNT; (b) acidic Th-based LPP monomer thiophene-3-yl acetic acid.

It is another object of the present invention to provide the method as defined above, wherein said step of Liquid Phase Polymerization (LPP) oxidative deposing said polymers is performed by at least one selected from a group consisting of (i) former type of “nucleophilized” Th-containing MWCNTs; (ii) acidic Th-based LPP monomer thiophene-3-yl acetic acid; (iii) Pyr/polyPyr; (iv) Pyr/polyPyr; and any combination thereof.

It is another object of the present invention to provide the method as defined above, wherein said step of Liquid Phase Polymerization (LPP) oxidative deposing said polymers is performed while using cationic cetyltrimethylammonium bromide (CTAB) concentration in the rang of about 0.01 to about 0.1 M for at least 1 hour.

It is another object of the present invention to provide the method as defined above, additionally comprising step of selectively depositing oxidative Liquid Phase Polymerizations (LPPs) onto the carbon nanotubes (CNT) surface is performed in a Liquid Phase Polymerization conditions (LPP conditions) selected from a group consisting of (a) concentration of cationic cetyltrimethylammonium bromide surfactant (CTAB) in the range of about 0[M] to about 0.1M; (b) the amount of Thiophene-3-yl acetic acid is in the range of 10.0 mg to about 35 mg; (c) the amount of Thiophene-3-yl acetic acid is in the range of 0.01 mmol to about 0.2 mmol; (d) the amount of Oxidant is in the range of 1.0 equiv./Th-monomer to about 3.5 equiv./Th-monomer; (e) the amount of Monomer solvent is in the range of 1.0 mL to about 3.5 mL; (1) Temp. of polymerization is in the range of 0 degrees to about 10 degree; (g) Time of polymerization is in the range of 0.5 hours to about 2 hours; and any combination thereof.

It is another object of the present invention to provide the method as defined above, wherein said Oxidant is Anhydrous FeCl₃.

It is another object of the present invention to provide the method as defined above, wherein said Monomer solvent is Distilled CHCl₃

It is another object of the present invention to provide a Th-decorated oxidized MWCNTs for use as nucleophilic nanosized phases in Liquid Phase Polymerization.

It is another object of the present invention to provide the Th-decorated oxidized MWCNTs as defined above, wherein said nucleophilic nanosized phases in Liquid Phase Polymerization is provided by the use of Thiophene (Th)-acetic acid precursor for polyCOOH polyTh-CP polymer deposition and covalent attachment.

It is another object of the present invention to provide the Th-decorated oxidized MWCNTs as defined above, wherein said decorative oxidized MWCNTs is provided in predetermined locations selected from sidewall, end-decorated, selectively end-decorated Th-CNTs and any combination thereof.

It is another object of the present invention to provide the Th-decorated oxidized MWCNTs as defined above, adapted to provide polymeric CP-chains grown oxidatively in bulk media of oxidative Liquid Phase Polymerizations (LPPs).

It is still an object of the present invention to provide the Th-decorated oxidized MWCNTs as defined above, adapted to provide a selective deposition onto at least one selected from a group consisting of the CNT surface, the CNT sidewall, or at oxidized extremities.

It is lastly an object of the present invention to provide the Th-decorated oxidized MWCNTs as defined above, wherein said selective deposition is provided at controlled amount and surface coverage.

### BRIEF DESCRIPTION OF THE FIGURES

In order to understand the invention and to see how it may be implemented in practice, a plurality of embodiments will now be described, by way of non-limiting examples only, with reference to the accompanying drawings, in which

FIG. 1 illustrates FT-IR spectrum of the polyCOOH polyTh-MWCNT composite showing characteristic vibration peaks and weight losses/temperature ranges

FIG. 2 illustrates TGA graph of the polyCOOH polyTh-MWCNT composite showing characteristic vibration peaks and weight losses/temperature ranges.

FIG. 3 illustrates the structures of Pyr/Cbz/Th-containing oxidizable monomers.

FIGS. 4-5 illustrate the fabrication processes and structures of Pyr/Cbz/Th-containing oxidizable monomers used for LPP deposition of corresponding CP phases onto sidewalls or selectively at oxidized extremities of pegylated oxidized MWCNTs.

FIG. 6 illustrates the TEM microphotographs of polyTh-MWCNT composite, sidewall and end polyTh deposition

FIGS. 7a-8 illustrate SEM & TEM microphotographs of pegylated polyTh-MWCNT composites, selective end-localized deposition of the polyTh phase.

FIGS. 9a-9b illustrate AFM images of polyCOOH polyTh-MWCNT dual phase composites showing the LPP deposition of the polyCOOH polyTh phase on MWCNT sidewalls.

FIGS. 10-13 illustrate TEM/SEM analyses of starting and oxidized MWCNTs and c-MWCNTs.

FIGS. 14a-14c illustrating the thermogravimetric analyses (TGA) of poly(3-4a,2b)/d-MWCNTs1-2a,1b composites.

FIG. 15 illustrates high resolution SEM (left) and high resolution TEM (right) microphotographs of dual phase composites poly(3a)/d-MWCNTd1a (a & b), poly(4a)/d-MWCNTd2a (c & d), and poly(2b)/d-MWCNTd1b (e & f).

FIG. 16 illustrates AFM images of polyCOOH c-MWCNTs (a), and of dual phase composites poly(3a)/d-MWCNTd1a (b), poly(4a)/d-MWCNTd2a (c), and poly(2b)/d-MWCNTd1b (d).

FIGS. 17a-17c illustrates statistical analysis of LPP outcome data: (a) normal probability plot of standardized effects, (b) Pareto chart of standardized effects, and (c) contour plot of deposited polyPyr-poly(4a) amounts (%) versus amounts of starting 4a monomer (mg) and LPP polymerization time (h).

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide Oxidized polycarboxylated Multi/Single/Double-walled Carbon Nanotubes (MW/SW/DW CNTsₒₓ) covalently functionalized by low molecular weight polythiophene (polyTh) monomeric precursors. These precursors contain an oxidizable thiophenyl (Th)-heterocycle.

The term “about” refers hereinafter to a range of 25% below or above the referred value.

The terms “1a”, “1b”, and “2a” refers herein after to polypyrrole/carbazolyl (Pyr/Cbz)-linkers as described in Scheme 1, reproduced bellow. The terms “3a”, “4a”, and “2b” refers herein after to oxidizable Pyr/Cbz-LPP monomers as described in Scheme 1, reproduced bellow.

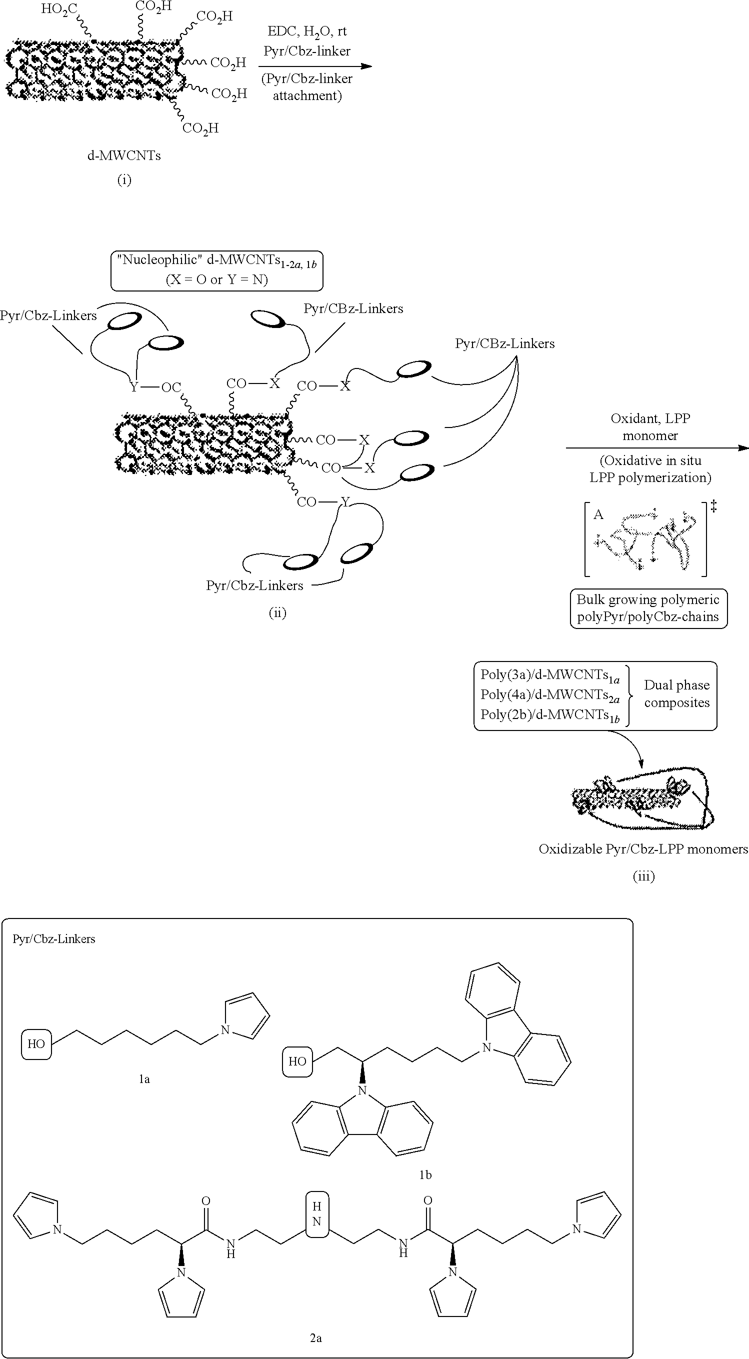

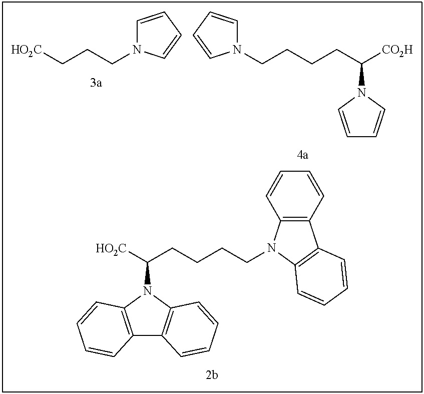

The present invention provides Oxidized polycarboxylated Multi-Walled Carbon Nanotubes (MWCNTs) ‘decorated’ by covalently attached pyrrolyl (Pyr)/carbazolyl (Cbz)-containing ligands.

This would result in a chemical modified MWCNTs that can be exploited as “nucleophilic” nanosized phases that enabled the covalent trapping of polymeric positively charged polyPyr/polyCbz-chains grown oxidatively in bulk media of oxidative Liquid Phase Polymerizations (LPPs).

The present invention further provides a two-step sequence for the controlled fabrication of functional (polyCOOH) and dual phase polyPyr/polyCbz-MWCNT composites provides a general solution to observed detrimental issues of phase compatibility during component assembly.

The present invention also investigated multi-parameter oxidative LPP process by using statistically relevant Design Of Experiments (DOE) so as to disclose influential LPP parameters towards process optimization.

As described above, the present invention provides a new methodological concept so as to overcome the eliminatation of polyPyr-poly(4a) and polyCbz-poly(2b) doped polymers having been during composite purification.

And thus, providing a general solution that will address the inherent phase compatibility issue cited above.

As described above, the core concept behind the method of the present invention are the following main steps:
(a) the Carbon Nanotube (CNT) oxidation;
(b) the linker (Pyr/Cbz and Th-based ones) covalent attachment (by COOH activating the polyCOOH shell);
(c) the polymer growth from the surface using the oxidative LPP protocol for growing conducting polymers of all the series of polypyrrole, polycarbazole and polythiophene.

The innovative concept behind the present invention combines two key steps, i.e. (i) first, covalently coupling/grafting oxidized polycarboxylated MWCNTs (c-MWCNTs) with hydroxylated or aminated Pyr/Cbz-containing linkers 1a-b and 2a (see scheme1 below, bottom left) affording Pyr/Cbz-decorated d-MWCNTs; and (ii), second, in-situ oxidatively polymerizing various functional monocarboxylated Pyr/Cbzmonomers 3-4a and 2b (LPP conditions), (see scheme below, bottom right) in the presence of former d-MWCNTs

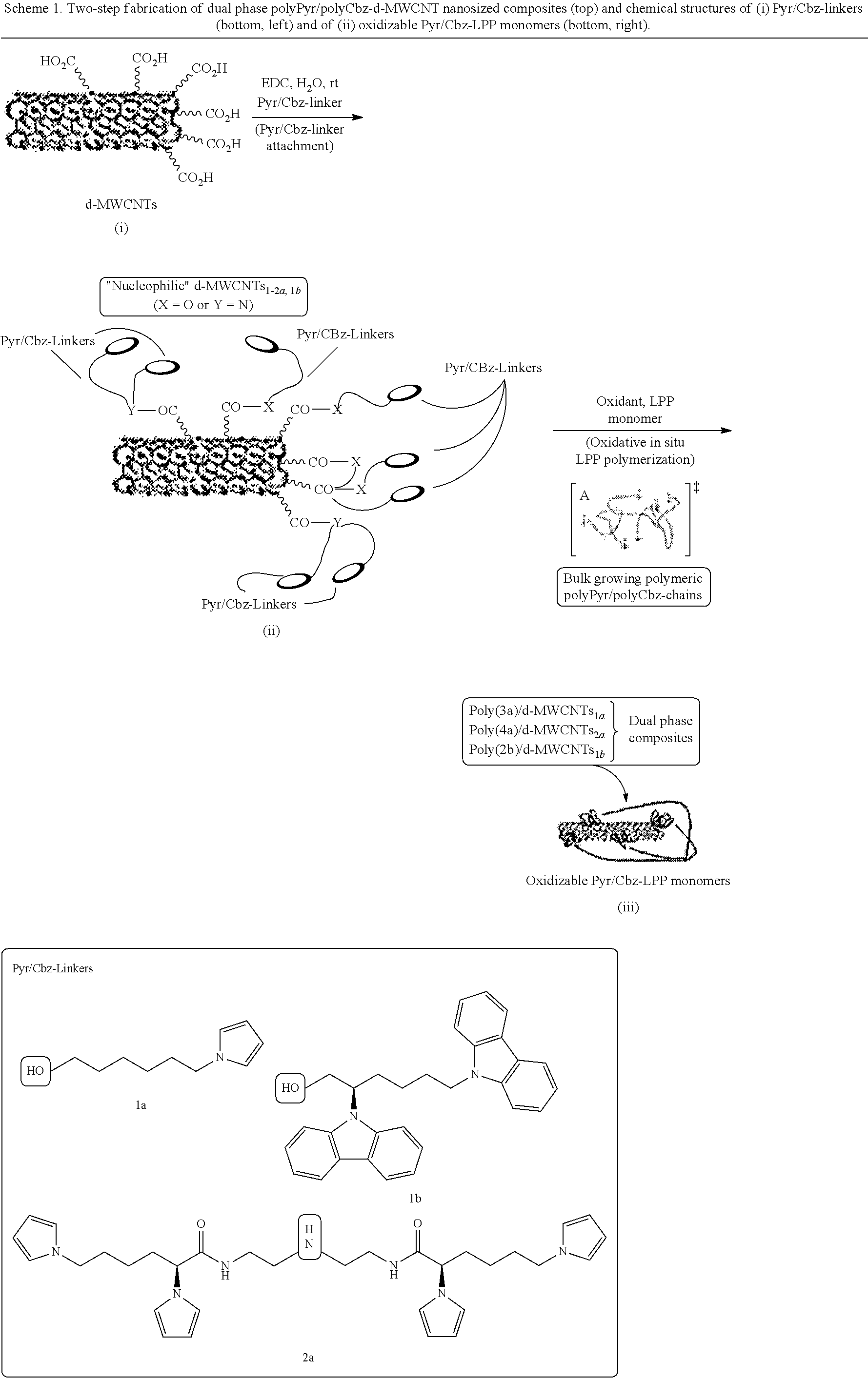

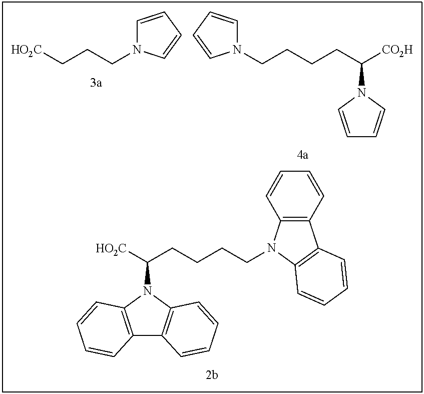

As described in the scheme above, first MWCNTs [MER Corporation Ltd., USA, —they were produced by chemical vapor deposition (CVD) and possess average diameters/lengths of 140±30 nm/7±2 nm respectively. They are composed of 340-530 graphitic layers and disclose purity higher than 90% as determined by thermogravimetric analysis (TGA)]-, are oxidized using a known wet-chemistry protocol, i.e., the use of an oxidative acidic 1/1 v/v mixture of concentrated 12M HNO₃ and 36M H₂SO₄ (70° C., 2 h) followed by multiple rinsing with bi-distilled H₂O until neutrality. It resulted in both simultaneous carboxylative opening of oxidation-sensitive end-caps (polyCOOH end cluster), and in the introduction of defect carboxylic (COOH) groups on sidewall surfaces of oxidized COOH-MWCNTs (see numerical reference (i) in scheme 1).

In a 2^{nd} step, the COOH activation of the polyCOOH shell (namely, out surface) has been executed using aqueous N′-(3-dimethylaminopropyl)-N-ethyl-carbodiimide (EDC) followed by the covalent attachment of the indicated Thp-containing linker thiophene-3-ethanol [1.0 mg MWCNT-COOH, 3.0 mg (15.7 mmoles) of EDC, 1.0 mL H₂O, 1 h, room temperature; then linker addition, 1.0 equiv./EDC in 1.0 mL CH₃CN, overnight, room temperature]. EDC reacts with MWCNT carboxylic acid groups to form an active O-acylisourea intermediate that has been easily displaced by nucleophilic attack using the corresponding hydroxylated Th-containing linker (see numerical reference (ii) in scheme 1). The alcohol moiety forms an ester bond with the activated carboxyl groups affording intermediate polyTh-decorated oxidized MWCNTs. The same polyCOOH activation step may be also successfully preformed using PEG-passivated oxidized MWCNTs (α,ω-bis-methoxy PEG_{2,000} polymer, Shearwaters Polymers, USA, MW=2,000 Daltons, 30.0 mg/3.0 mL of distilled water; 200 μL, 20 min incubation, 20° C.) in order to afford end-selectively Th-decorated oxidized MWCNTs.

The last fabrication step basically consisted in the LPP oxidative deposition of the polyCOOH polyTh-CP polymers using (i) either former type of “nucleophilized” Th-containing MWCNTs (sidewall/end Th-decorated or selectively PEG-passivated end-decorated Th-containing MWCNTs) and (ii) the acidic Th-based LPP monomer thiophene-3-yl acetic acid (see scheme 1). Optimized LPP conditions are summarized below being applied to (i) Th-decorated oxidized MWCNTs or to (ii) pegylated sidewall passivated oxidized MWCNTs (end selective growth of the 2^{nd} polyTh-phase), see numerical reference (iii) in scheme 1.

As a matter of consequence, this intermediate d-MWCNT1-2a,1b phase will now compete with bulk Pyr/Cbz-monomers for CP chain elongation.

It will act as a “nucleophilic” nanosized phase able to trap the cation/radical electrophilic polyPyr/polyCbz-CP polymer chains of type A (see scheme 1) generated in the bulk medium during LPP experiments.

In this way, corresponding functional polycarboxylated (polyCOOH) polyPyr/polyCbz-polymers will be grown and deposited onto d-MWCNTs1-2a,1b leading to novel CP/CNT biphasic composite materials poly(3-4a,2b)/d-MWCNTs1-2a,1b. In addition, underlayer weight depositions of CP polymers may be strictly controlled using unique sets of LPP rectional parameters optimized by statistically relevant Design Of Experiments (DOE). In order to demonstrate this, the illustrative case of the DOE-optimized preparation of the poly(4a)/d-MWCNTs2a composite has been reported below as part of these studies. Consequently, morphologically versatile CP/CNT composite materials that contained controlled weight ratios of both CP/CNT phases have been readily fabricated applying this LPP methodological variant.

The present invention also provides a novel LPP-mediated methodological concept for the fabrication of functional (polyCOOH) dual phase polyPyr/polyCbz-MWCNT composites has been validated. Accordingly, oxidative LPP experiments involved appropriate acidic Pyr/Cbz-containing monomers 2b, 3a-4a (see scheme 1) and, more importantly “nucleophilic” Pyr/Cbz-modified oxidized MWCNTs (d-MWCNTs) instead of untreated MWCNTs.

Due to their intrinsic designed nucleophilicity (pending Pyr/Cbz-heterocycles), such “nucleophilic” Pyr/Cbz-modified d-MWCNTs successfully competed with similar bulk Pyr/Cbzmonomers toward in situ generated electrophilic growing CP chains. As a matter of consequence, this two-step LPP process enabled a full control of the morphologies of resulting dual phase polyPyr/polyCbz-MWCNT composites (polymer deposition onto the d-MWCNT surface). Moreover, a quite attractive feature of this innovative LPP variant was to readily solve the detrimental issue of phase compatibility during CP and MWCNT phase assembling and/or interaction. Relating to the fabrication of the poly(4a)/d-MWCNTs2a composite, this LPP variant has been also investigated regarding influential parameters and potential parameter synergism using a statistically relevant Design Of Experiments (DOE) method. Two main conclusions of this DOE-mediated study have been drawn.

First and in the proposed range of evolution of process parameters (4a amount, CTAB concentration, LPP reaction time) specific sets of LPP conditions provided optimal depositions of the polyPyr-poly(4a) polymer in a 17.7-18.1% weight range. Second, only one influential LPP parameter, i.e. the amount of oxidized Pyr-4a monomer has been identified.

The above mentioned chemically modified Th-decorated CNTs may contain the Th-decoration either at sidewalls and/or oxidized extremities selectively if using an intermediate passivating PEG polymer polyCOOH protection step. Both types of Th-decorated oxidized MWCNTs, meaning sidewall/end-decorated or selectively end-decorated Th-CNTs have been successfully exploited as novel nucleophilic nanosized phases in Liquid Phase Polymerization experiments using a Thiophene (Th)-acetic acid precursor for polyCOOH polyTh-CP polymer deposition and covalent attachment.

Therefore, polymeric CP-chains grown oxidatively in bulk media of oxidative Liquid Phase Polymerizations (LPPs) may be selectively deposited onto the CNT surface (sidewall) or at oxidized extremities (in the case of the PEG polymer passivation) at controlled amount and surface coverage.

This two-step sequence for the fabrication of functional dual phase CP (polyTh)-CNT composites constitutes a general solution to observed detrimental issues of phase compatibility occurring in a recurrent manner during component assembly of such entities.

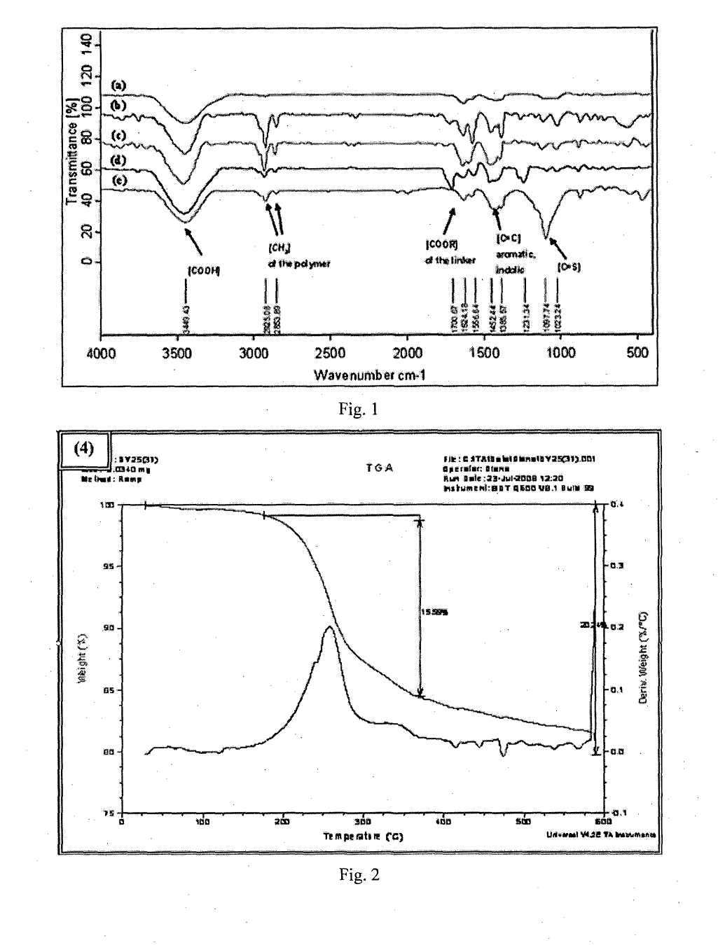

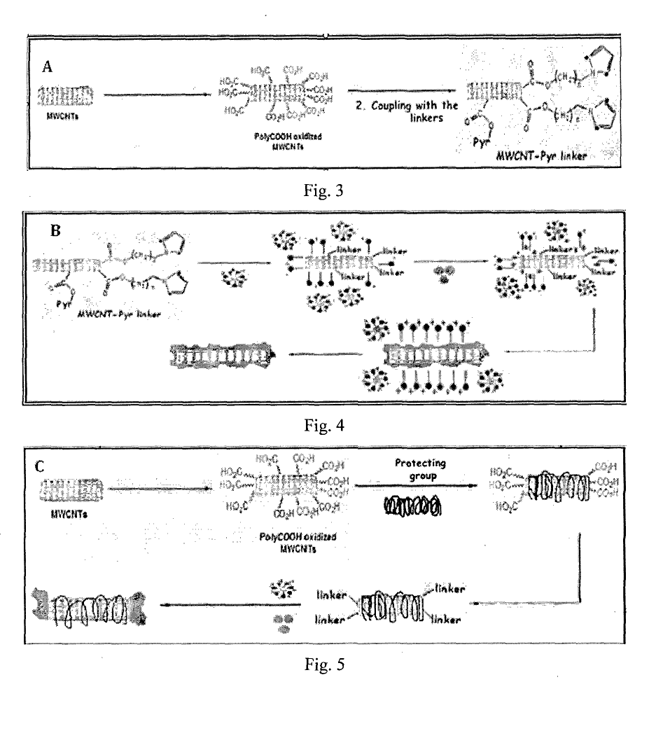

More specifically, the present invention discloses two main steps involved in the fabrication of both types of polyTh-MWCNT composites using an innovative “growth from” method in Liquid Phase Polymerization conditions (LPP conditions). First MWCNTs [obtained from MER Corporation Ltd., USA, said MWCNT were produced by chemical vapor deposition (CVD) and possess average diameters/lengths of 140±30 nm/7±2 nm respectively. They are composed of 340-530 graphitic layers and disclose purity higher than 90% as determined by thermogravimetric analysis (TGA)]-, are oxidized using a known wet-chemistry protocol, i.e., the use of an oxidative acidic 1/1 v/v mixture of concentrated 12M HNO₃ and 36M H₂SO₄ (70° C., 2 h) followed by multiple rinsing with bi-distilled H₂O until neutrality. It resulted in both simultaneous carboxylative opening of oxidation-sensitive end-caps (polyCOOH end cluster), and in the introduction of defect carboxylic (COOH) groups on sidewall surfaces of oxidized COOH-MWCNTs. Reference is now made to FIGS. 1-2, illustrating the FT-IR spectrum (FIG. 1) and TGA graph (FIG. 2) of the polyCOOH polyTh-MWCNT composite showing characteristic vibration peaks and weight losses/temperature ranges.

In the 2^{nd} step, the COOH activation of the polyCOOH shell has been executed using aqueous N′-(3-dimethylaminopropyl)-N-ethyl-carbodiimide (EDC) followed by the covalent attachment of the indicated Thp-containing linker thiophene-3-ethanol [1.0 mg MWCNT-COOH, 3.0 mg (15.7 mmoles) of EDC, 1.0 mL H₂O, 1 h, room temperature; then linker addition, 1.0 equiv./EDC in 1.0 mL CH₃CN, overnight, room temperature].

EDC reacts with MWCNT carboxylic acid groups to form an active O-acylisourea intermediate that has been easily displaced by nucleophilic attack using the corresponding hydroxylated Th-containing linker (see FIGS. 1-2).

The alcohol moiety forms an ester bond with the activated carboxyl groups affording intermediate polyTh-decorated oxidized MWCNTs.

The same polyCOOH activation step may be also successfully preformed using Polyethylene glycol (PEG) PEG-passivated oxidized MWCNTs (α,ω-bis-methoxy PEG_{2,000} polymer, Shearwaters Polymers, USA, MW=2,000 Daltons, 30.0 mg/3.0 mL of distilled water; 200 μL, 20 min incubation, 20° C.) in order to afford end-selectively Th-decorated oxidized MWCNTs.

The last fabrication step basically consisted in the Liquid Phase Polymerization (LPP) oxidative deposition of the polyCOOH polyTh-CP polymers using (i) either former type of “nucleophilized” Th-containing MWCNTs (sidewall/end Th-decorated or selectively PEG-passivated end-decorated Th-containing MWCNTs) and (ii) the acidic Th-based LPP monomer thiophene-3-yl acetic acid (as used in FIGS. 1-2). Optimized LPP conditions are summarized below being applied to (i) Th-decorated oxidized MWCNTs or to (ii) pegylated sidewall passivated oxidized MWCNTs (end selective growth of the 2^{nd} polyTh-phase). Each differently decorated nucleophilic MWCNT-Th-linker composite (25.0 mg) was separately suspended in distilled CHCl₃ (8 mL) in the presence of CTAB (cationic cetyltrimethylammonium bromide surfactant, 364.5 mg, 0.1M final concentration).

A 1 h-long ultrasonication using a Bransonic® bath sonicator (42 KHz at full power) afforded well-dispersed suspensions of each corresponding CTAB/MWCNT-Th-linker material.

Then, to the related CTAB/MWCNT-Th-linker-based dispersions, the previously dissolved organic acidic Th-based LPP monomer thiophene-3-yl acetic acid was slowly added dropwise, followed by the addition of the indicated LPP oxidant (FeCl₃) as a neat powder (see table 1):

TABLE 1
LPP experiments involving Th-monomer-decorated oxidized MWCNTs

| | | | Thiophene-3-yl | Oxidant | Monomer | Time & |
|---|---|---|---|---|---|---|
| | MWCNT | CTAB | acetic acid | (equiv./Th- | solvent | Temp. of |
| Entry | Type | [M] | (mg, mmol) | monomer) | (mL) | polymerization |
| 1 | MWCNT- | — | 20.0, 0.14 | Anhydrous | Distilled | 2 h & 0° C. |
| | Th-linker | | | FeCl₃/2.5 eq | CHCl₃ | |
| | (thiophenyl-3- | | | | (2 mL) | |
| | ethanol) | | | | | |
| 2 | MWCNT- | 0.01M | 20.0, 0.14 | Anhydrous | Distilled | 2 h & 0° C. |
| | Th-linker | | | FeCl₃/2.5 eq | CHCl₃ | |
| | (thiophenyl-3- | | | | (2 mL) | |
| | ethanol) | | | | | |
| 3 | MWCNT- | 0.1M | 20.0, 0.14 | Anhydrous | Distilled | 2 h & 0° C. |
| | Th-linker | | | FeCl₃/2.5 eq | CHCl₃ | |
| | (thiophenyl-3- | | | | (2 mL) | |
| | ethanol) | | | | | |
| 4 | Pegylated | 0.1M | 20.0, 0.14 | Anhydrous | Distilled | 2 h & 0° C. |
| | MWCNT- | | | FeCl₃/2.5 eq | CHCl₃ | |
| | Th-linker | | | | (2 mL) | |
| | (thiophenyl-3- | | | | | |
| | ethanol) | | | | | |

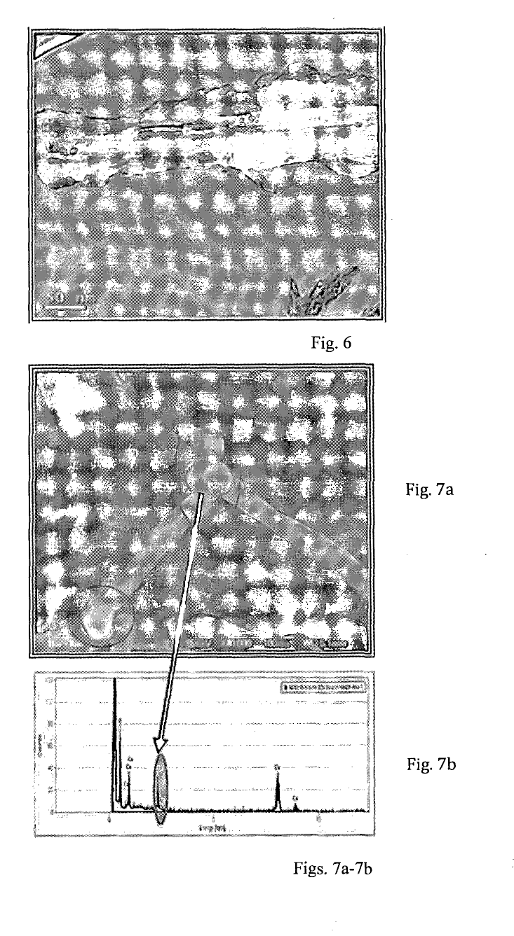

Reference is now made to FIG. 3 illustrating the structures of Pyr/Cbz/Th-containing oxidizable monomers.

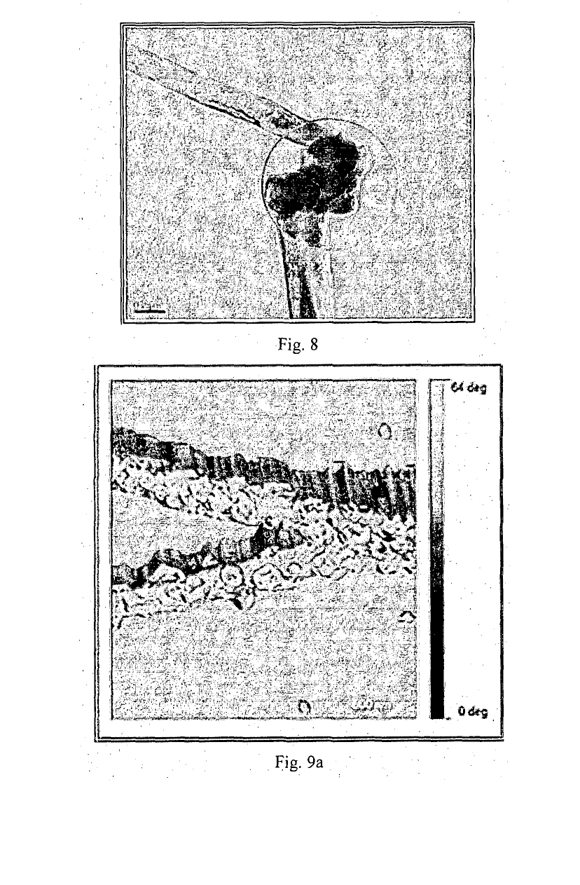

FIGS. 4-5 illustrate the fabrication processes (FIG. 4-5) and structures of Pyr/Cbz/Th-containing oxidizable monomers (FIG. 3) used for LPP deposition of corresponding CP phases onto sidewalls (FIG. 4) or selectively at oxidized extremities of pegylated oxidized MWCNTs (FIG. 5).

The following description provides characterization of polyCOOH polyTh-MWCNT composites (sidewall/end and selectively end-deposited CP phase).

A wide range of analytical, spectroscopic, and microscopy methods have been used in order to fully characterized both types of polyCOOH polyTh-MWCNT composites possessing both morphologies, i.e., deposition of the polyTh-phase onto the sidewall/end and selectively at oxidized extremities (pegylated sidewall passivated oxidized MWCNTs).

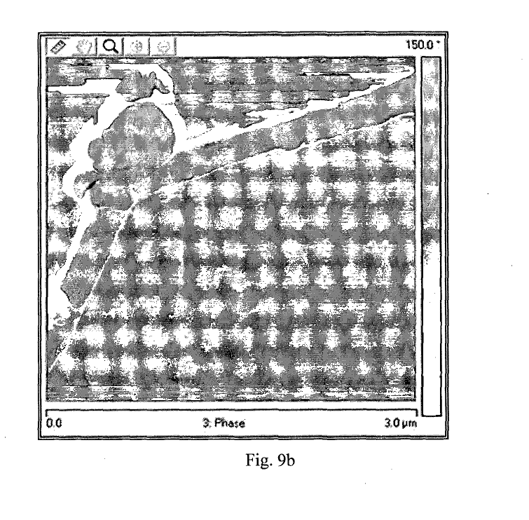

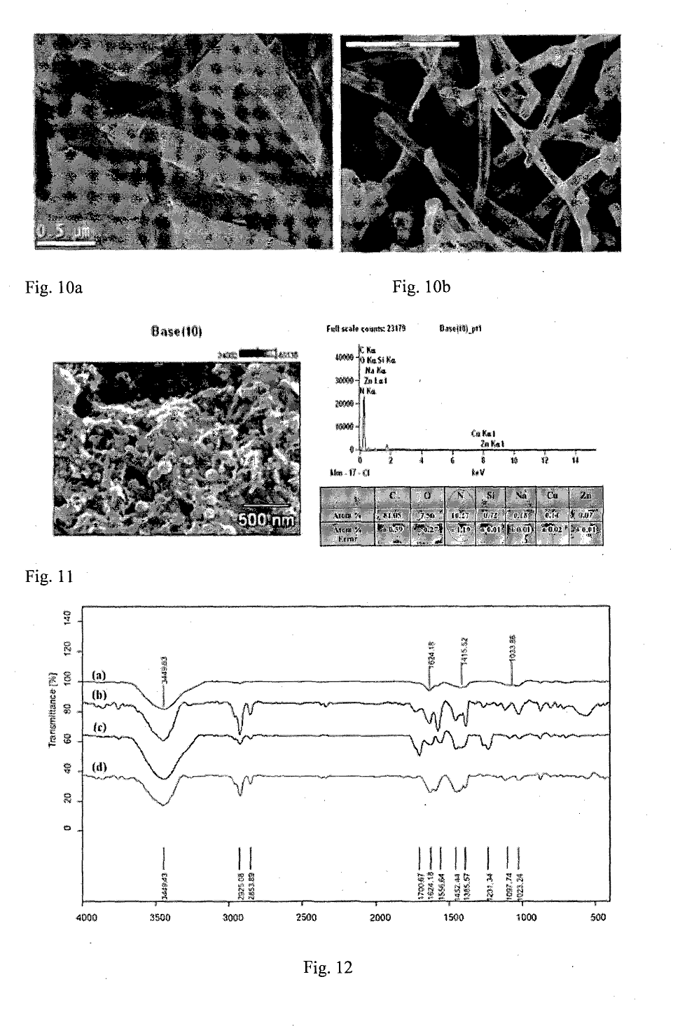

Reference is now made to FIGS. 6-7b which illustrates TEM and SEM microphotographs of polyCOOH polyTh-MWCNT dual phase composites.

FIG. 6 illustrates the TEM microphotographs of polyTh-MWCNT composite, sidewall and end polyTh deposition

FIGS. 7a-8 illustrate SEM & TEM microphotographs of pegylated polyTh-MWCNT composites, selective end-localized deposition of the polyTh phase.

FIG. 7b discloses the EDS compositional analysis of the pegylated end-polyTh-functionalized polyTh-MWCNT composite (S element detection).

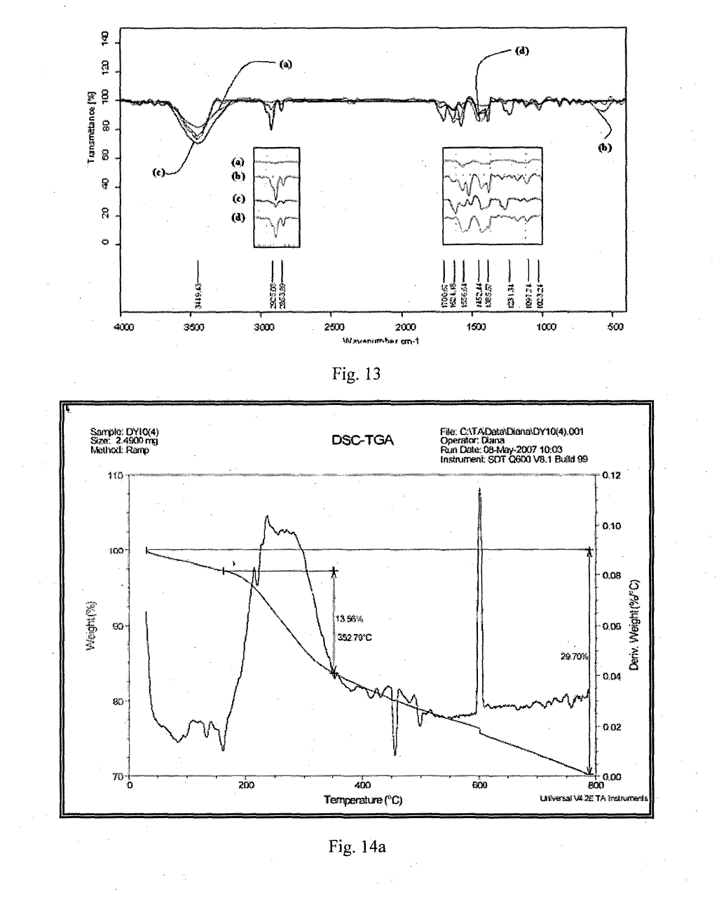

Reference is now made to FIGS. 9a-9b which illustrate AFM images of polyCOOH polyTh-MWCNT dual phase composites showing the LPP deposition of the polyCOOH polyTh phase on MWCNT sidewalls.

FIG. 9a illustrates AFM images of polyCOOH polyTh-MWCNT composite and at extremities of oxidized PEG-passivated MWCNTs.

FIG. 9b illustrates AFM images of pegylated end-functionalized polyCOOH polyTh-MWCNTs.

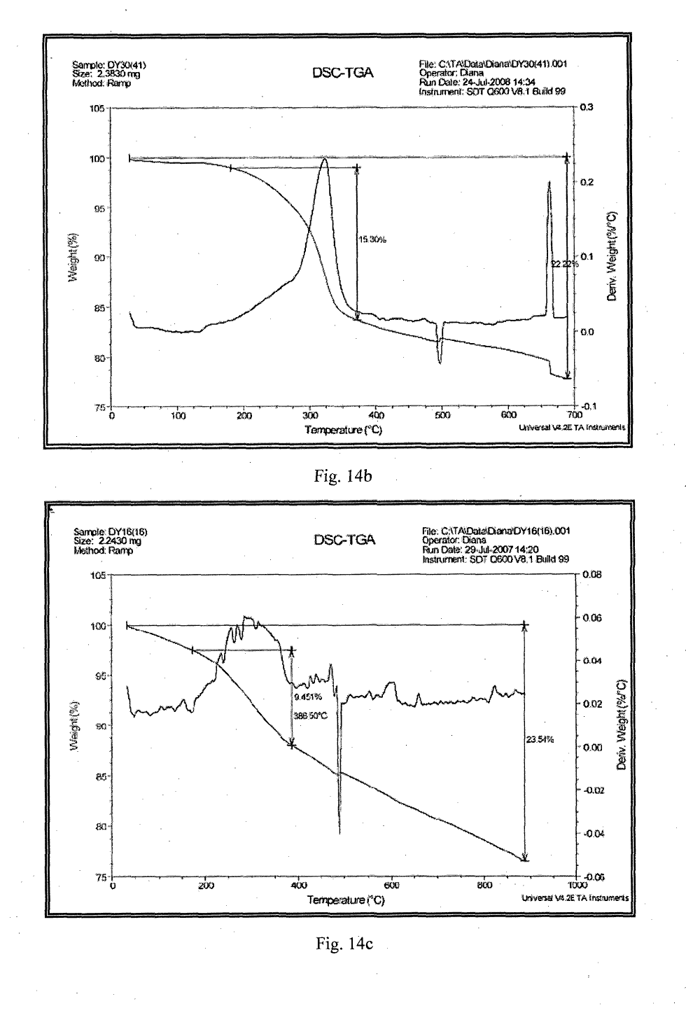

According to another embodiment of the present invention, LPP methodological variant, Pyr/Cbz-decorated d-MWCNTs1-2a,1b that acted as competitive “nucleophilic” nanomaterial phases versus bulk Pyr/Cbz-containing monomers 3-4a and 2b were expected to lead to the controlled deposition of insoluble doped poly(3-4a,2b) CP polymers onto d-MWCNTs1-2a,1b sidewalls. Subsequently, preliminary FT-IR (FT-IR Braker Equinox 55 spectrometer, 1% weight KBr dispersion pellets) spectroscopic and TGA (Thermofinnigan TA Q600-0348, model SDT Q600, temperature profile: 50-800° C. at 20° C./min under N2) characterizations of resulting CP/CNT composite materials have been performed aiming at checking the presence of organic polyPyr/polyCbz-CP phases. All the analyzed poly(3-4a,2b)/d-MWCNTs1-2a,1b composites showed characteristic FT-IR peaks proving coherent chemical functionality Reference is now made to FIGS. 10-13 illustrating TEM/SEM analyses of starting and oxidized MWCNTs and c-MWCNTs.

FIGS. 10a-10b illustrate High-resolution TEM microphotographs of starting MWCNTs (MER Corporation Ltd., FIG. 10a) and low-resolution SEM microphotographs of corresponding polyCOOH c-MWCNTs (FIG. 10b) isolated from LPP experiments involving oxidizable LPP Pyr- and Cbz-based monomers 4a & 2b.

FIG. 11 Illustrates SEM microphotograph and compositional EDAX analysis of bulk poly(4a) obtained from the washing phase of LPP experiments involving Pyr-monomer 4a and non-modified MWCNTs and/or c-MWCNTs (emphasis on the presence of the N element).

FIGS. 12-13 illustrate FT-IR spectroscopy of c-MWCNTs and of poly(3-4a,2b)/d-MWCNTs1-2a,1b composites.

FIGS. 12-13 illustrate the combined FT-IR spectra of starting polyCOOH c-MWCNTs (a) and of the three composites poly(3a)/d-MWCNTd1a (b), poly(2b)/d-MWCNTd1b (c), and poly(4a)/d-MWCNTd2a (d) emphasizing corresponding functional groups.

Typical vibration peaks were found such as (a) vOH stretching peaks in the 3449.4-3451.0 cm-1 zone (polyCOOH cluster functionality, strong & large), (b) νCsp3-H stretching peaks at 2853.8-2854.6/2925.1-2925.0 cm-1

(medium to strong, alkyl chains), (c) νC═O stretching peaks at 1576.1-1700.1 cm-1 (polyCOOH group) and (d) aromatic/indolic C═C double bond stretching vibration peaks for both doped π-conjugated polyPyr and polyCbz systems (1385.6-1454.3 and 1452.4-1456.4 cm-1 zones respectively, strong).

Thermogravimetric analyses (TGA) analysis also demonstrated the presence of corresponding polymeric polyPyr/polyCbz-phases.

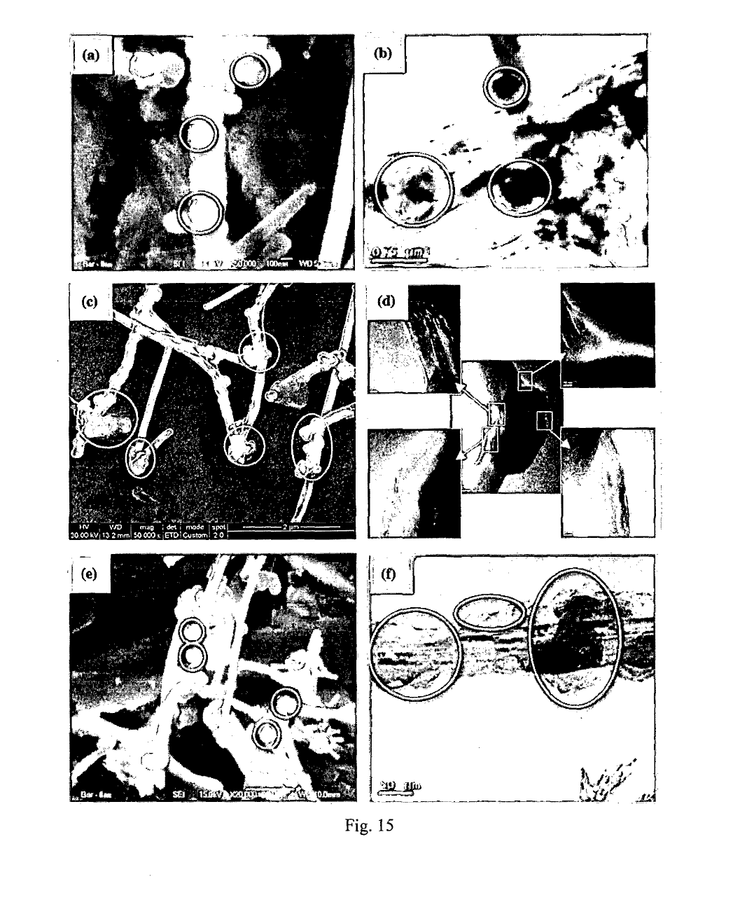

Reference is now made to FIGS. 14a-14c illustrating the thermogravimetric analyses (TGA) of poly(3-4a,2b)/d-MWCNTs1-2a,1b composites.

The thermogravimetric (TGA) graphs of poly(3-4a,2b)/d-MWCNTs1-2a,1b composites shows % of weight losses versus increasing temperatures.

FIG. 14a illustrates the Poly(3a)/d-MWCNTs1a TGA.

FIG. 14b illustrates the Poly(4a)/d-MWCNTs2aTGA.

FIG. 14c illustrates the Poly(2b)/d-MWCNTs1bTGA.

Both polyPyr-based composites poly(3a)/d-MWCNTs1a and poly(4a)/d-MWCNTs2a disclosed similar one-step 13.6 and 15.3% weight losses respectively corresponding to the polyPyrphase burning/decomposition in a 180-352.8° C. temperature range. In contrast, the more difficultly LPP oxidized polyCbz-based27 poly(2b)/d-MWCNTs1b composite registered a minored one-step 9.5% weight loss corresponding to the polyCbz-phase burning/decomposition (190-386.5° C. temperature range).

Corresponding weight losses and decomposition temperature data correlated those obtained for 20-40 nm-sized magnetically responsive polycarboxylated magnetite-polyPyr/polyCbz-based nanocomposites of a core (magnetite)-shell (polyPyr/polyCbz) morphology. These magnetic biphasic composite nanoparticles were produced using structurally similar acidic Pyr/Cbz-monomers oxidatively deposited by a same ultrasound-assisted LPP procedure.

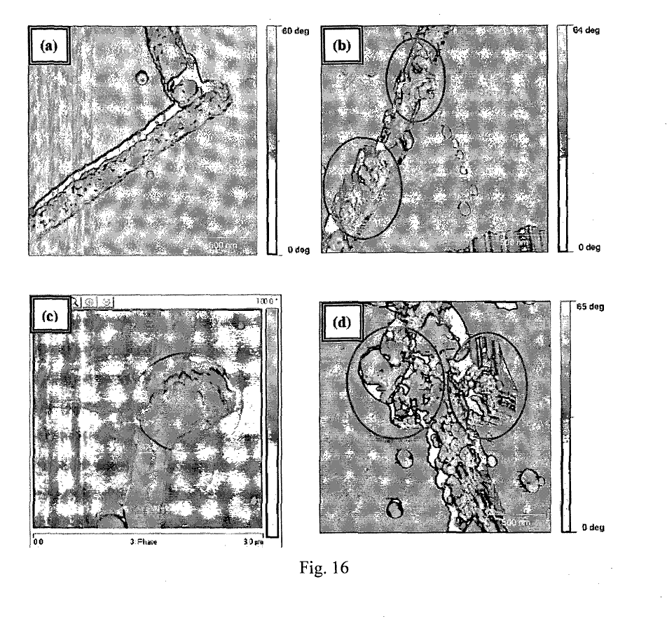

All these composites were then examined by high resolution HR-SEM (JEOL JSM-7000P apparatus, Oxford Instruments, Gatan CCD Camera, without Au evaporation) and HR-TEM (FEI Titan 80-300 kV, accelerating voltage 300 keV, Gatan CCD camera). Both types of microphotographs (see FIG. 15) showed the presence of globular polyPyr/polyCbz-based polymeric excrescences irregularly deposited around MWCNT sidewalls (see the indicated yellow circles, FIG. 15).

FIG. 15 illustrates high resolution SEM (left; namely, FIGS. 15a, 15c and 15e) and high resolution TEM (right; namely, FIGS. 15b, 15d and 15f) microphotographs of dual phase composites poly(3a)/d-MWCNTd1a (a & b), poly(4a)/d-MWCNTd2a (c & d), and poly(2b)/d-MWCNTd1b (e & f).

Resulting morphologies likely arose from sidewall-confined polyPyr/polyCbz-polymer growth caused by sidewall pending Pyr/Cbz-linkers that acted as nucleophilic trapping species. Furthermore and in comparison to average diameter sizes of starting MWCNTs (140±30 nm), diameters of MWCNTs within CP-modified poly(3-4a,2b)/d-MWCNTs1-2a,1b composites have been measured by HR-TEM and emphasized polymer deposition onto isolated MWCNTs or small two-fold associated MWCNT aggregates (Ø˜160-250 nm).

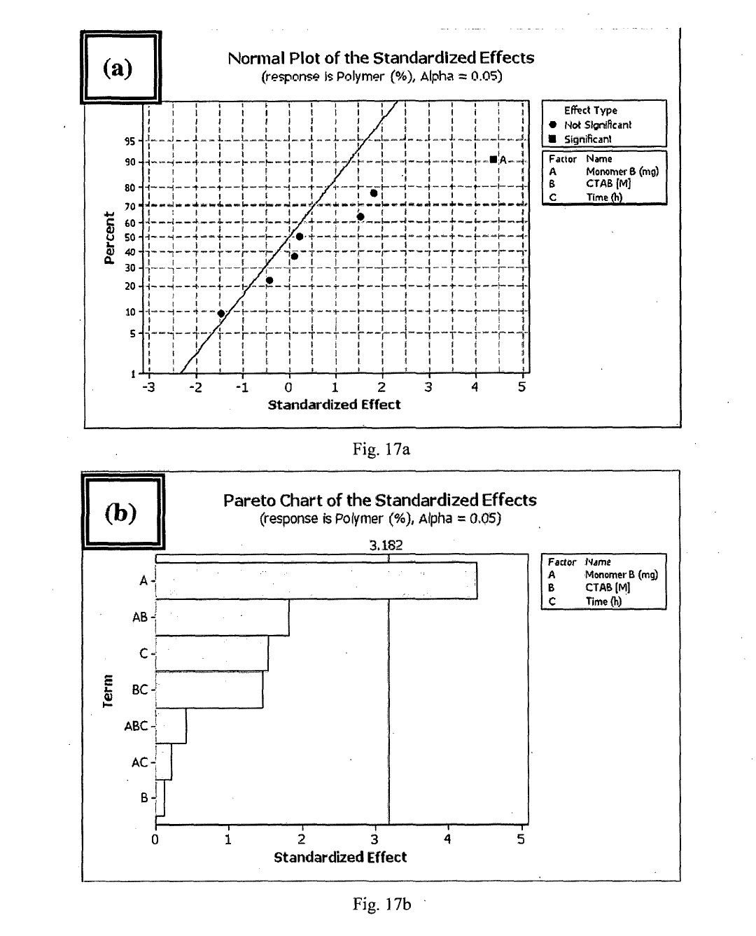

This last result is worthwhile to notice meaning that this oxidative ultrasound-assisted LPP system enabled almost total breaking-up of loosely aggregated d-MWCNT bundles during polyPyr/polyCbz-polymer LPP deposition. Validation of this novel methodological LPP process, i.e. the polyPyr/polyCbz-polymer growth from anchoring Pyr/Cbz-containing linkers attached onto d-MWCNT sidewalls has been also performed using AFM (Nanoscope V Multimode scanning probe microscope, tapping mode using a single PPP-NCL silicon probe (force constant of 21-98 N/m), see FIG. 16).

FIG. 16 illustrates AFM images of polyCOOH c-MWCNTs (a), and of dual phase composites poly(3a)/d-MWCNTd1a (b), poly(4a)/d-MWCNTd2a (c), and poly(2b)/d-MWCNTd1b (d).

Oxidized polyCOOH c-MWCNTs (FIG. 16a) presented a clear porous texture of MWCNT sidewalls resulting from the oxidative introduction of surface carboxyl defects. In contrast, all the three composites (FIG. 16b-d) showed similar under-layer irregular areas indicative of the presence of deposited covalently attached polymeric polyPyr/polyCbz-species (FIG. 16b-d, blue circled areas).

DOE-Mediated Preparation of the Poly(4a)/d-Mwcnts2a Composite Material

At this stage, the fabrication of the illustrative polyPyr-based poly(4a)/d-MWCNTs2a composite material has been the object of a further refinement study (disclosure of influential LPP parameters). In fact, the versatility of the LPP methodological variant using “nucleophilic” d-MWCNTs2a has been investigated for its capability to deliver various similarly shaped (under-layer polymer deposition onto MWCNT sidewalls) poly(4a)/d-MWCNTs2a composites that will contain controlled amounts of functional polyCOOH polyPyr/poly(4a)-polymers. Subsequent data may critically affect potential 2^{nd} step chemical modifications of such polyCOOH composites based of known activation chemistries of the carboxylate groups (carbodiimides or mixed anhydrides for example) present in deposited poly(3-4a,2b) polymers.

For that purpose and since the corresponding LPP process is multi-parametric in nature, the present invention addressed this issue using statistically relevant Design Of Experiments (DOE). Through simple experimental designs (see below), DOE-based approaches use powerful statistical tools that enable the identification of significant parameters and of possible phenomena of inter-parameter synergism. This unique feature combination generally led to the disclosure of unique globally optimized set(s) of reaction/process conditions via the obtainment of robust and reliable protocol(s). In this context, a three factor-two level full factorial design [(Res IV) 2³=8+3=11] experiments comprising a triplicate center point (see Table 2, Run Orders n° 3, 5, and 11)] was proposed by the Design-Expert software MINITAB15® (Minitab Inc., State College, Pa., USA).

The three LPP parameters that were investigated regarding LPP outcomes were (i) the amount of Pyr-monomer 4a at constant volume (4a, two values: 20.0 and 60.0 mg/0.081 and 0.243 mmol), (ii) the cationic cetyltrimethylammonium bromide (CTAB) surfactant concentration ([CTAB], two values: 0.01 and 0.1M), and (iii) the LPP oxidative polymerization time (oxidation time, two values: 1.0 and 2.0 h). In accordance with preliminary non-DOE-based LPP screening data (results not shown), the FeCl₃.6H₂O oxidant parameter that was found non-influential has not been included in the above factorial experiment design.

TABLE 2
Proposed and randomly executed DOE matrix of experiments
relating to the multi-parameter fabrication of the
poly(4a)/d-MWCNTs2a composite material

| Run | 4a | [CTAB] | Time | PolyPyr- |
|---|---|---|---|---|
| Order | (mg, mmol) | (M) | (h) | Poly(4a) (%) |
| 1 | 60.0-0.243 | 0.01 | 2.0 | 17.7 |
| 2 | 20.0-0.081 | 0.1 | 1.0 | 5.17 |
| 3^{a} | 40.0-0.162 | 0.055 | 1.5 | 8.7 |
| 4 | 20.0-0.081 | 0.01 | 2.0 | 11.3 |
| 5^{a} | 40.0-0.162 | 0.055 | 1.5 | 8.2 |
| 6 | 20.0-0.081 | 0.01 | 1.0 | 6.5 |
| 7 | 60.0-0.243 | ″ | 1 | 10.4 |
| 8 | ″ | 0.1 | ″ | 18.1 |
| 9 | ″ | ″ | 2 | 17.9 |
| 10 | 20.0-0.081 | ″ | ″ | 5.7 |
| 11^{a} | 40.0-0.162 | 0.055 | 1.5 | 7.9 |

^{a}Triplicate central point (see text below for details) showing the corresponding limited data dispersion in a 7.9-8.7% range

According to a proposed MINITAB15® cubic model, an additional center point (triplicate format) was also added that corresponded to the following set of conditions [4a amount: 40.0 mg (0.162 mmol), [CTAB]: 0.055M, and LPP polymerization time: 1.5 h, FeCl₃.6H₂O: 1.0 equiv./equiv. of monomer 4a].

These conditions were based on the effective LPP protocol as will be described (see Example Section).

Accordingly, a set of eleven corresponding experiments has been executed in a random manner as proposed in the DOE matrix of experiments described in Table 2. LPP outputs were the respective amounts of poly(4a) polymer deposits measured by thermogravimetry (TGA, weight losses registered in the 180-384.0° C. temperature range, see Table 2).

At a first glance, the amounts of deposited polyPyr-poly(4a) polymer were measured in a 5.2-18.1% range, which a fortiori validated this DOE refinement study.

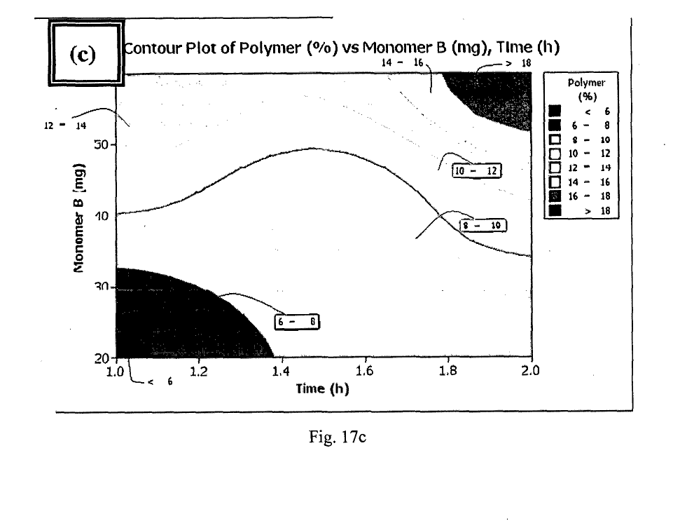

Reference is now made to FIGS. 17a-17c illustrating statistical analysis of LPP outcome data: (a) normal probability plot of standardized effects, (b) Pareto chart of standardized effects, and (c) contour plot of deposited polyPyr-poly(4a) amounts (%) versus amounts of starting 4a monomer (mg) and LPP polymerization time (h).

From both corresponding Normal Probability graph (FIG. 17a) and Pareto Chart (FIG. 17b), the statistical analysis of resulting LPP/TGA data showed that neither CTAB surfactant concentration nor polymerization time parameters had any effect on the amounts of deposited polyPyrpoly(4a) polymer for the tested evolution range of parameters. In contrast, the quantity of added Pyrmonomer4a has been found the sole most influential factor (classified as significant effect, FIG. 17a).

Interestingly, three sets of conditions have been disclosed that enabled highest most effective depositions of the polyPyr-poly(4a) polymer phase in a 17.7-18.1% range (see Table 2, Run Orders 1, 8-9). These conditions always used an optimal amount of 60.0 mg (0.243 mmol) of the Pyr-monomer 4a for a polymerization time of 1.0-2.0 h. This trend has been graphically represented in the corresponding Contour Plot of Polymer graph (see the relating dark-green area, FIG. 17c, in which the % polymer is greater than 18).

The present invention provided the full characterization proving the topologically selective deposition of the LPP polyTh phase has been provided emphasizing the exact morphologies of resulting composites.

Similar characterization works have been described in an article by Diana Goldman and J.-P. Lellouche, An easy method for the production of functional polypyrrole/MWCNT and polycarbazole/MWCNT composites using nucleophilic multi-walled carbon nanotubes, Carbon, 2010, 48, 4170-4177, fully incorporated within the present invention.

Thus, it is one object of the present invention to provide a “growth from” method for selectively depositing oxidative Liquid Phase Polymerizations (LPPs) onto the carbon nanotubes (CNT) surface, said method comprising steps of:
a. obtaining Multi-walled Carbon Nanotubes (MWCNT);
b. oxidized said MWCNTs to obtain oxidized COOH-MWCNTs; thereby (a) carboxylative opening oxidation-sensitive end-caps (polyCOOH end cluster); and, (b) introducing defect carboxylic (COOH) groups onto predetermined areas of said oxidized COOH-MWCNTs;
c. COOH activating the polyCOOH shell (namely, the surface) using various COOH activating species; and,
d. executing Liquid Phase Polymerization (LPP) oxidative deposing polymers selected from said polyCOOH polyTh-CP polymers, polyCOOH polyTh-, polyEDOT (PEDOT)-, polyTh polyCOOH poly(thiophenyl-3 acetic acid, thiophenyl-3 acetic acid/EDOT, polyX, wherein X is elected from COOH, OH, NH₂, polyCbz/polyPyr CP polymers and related combinatorial mixtures, polyCOOH PEDOT-poly(thiophenyl-3 acetic acid)′ thereby selectively depositing said oxidative LPPs onto said CNT surface.

It is another object of the present invention to provide the method as defined above, wherein said selectively deposition is performed in a controlled manner and for controlled polymer deposited amounts.

It is another object of the present invention to provide the method as defined above, wherein said predetermined area are selected from a group consisting of sidewall surfaces of said oxidized COOH-MWCNTs or CNT extremities or topologically selectively at only oxidized extremities of pegylated oxidized polyTh-decorated MWCNTs, end-decorated, selectively end-decorated Th-CNTs and any combination thereof.

It is another object of the present invention to provide the method as defined above, wherein said step of obtaining Multi-walled Carbon Nanotubes (MWCNT) is performed by chemical vapor deposition (CVD) and possess average diameters/lengths of 140±30 nm/7±2 nm respectively.

It is another object of the present invention to provide the method as defined above, wherein said MWCNT are composed of about 340 to about 530 graphitic layers and disclose purity higher than 90% as determined by thermogravimetric analysis (TGA)]; It is another object of the present invention to provide the method as defined above, wherein said step of oxidizing said MWCNT is performed by known wet-chemistry protocol.

It is another object of the present invention to provide the method as defined above, wherein said step of oxidizing said MWCNT by known wet-chemistry protocol is performed by steps of (a) oxidative acidic 1/1 v/v mixture of concentrated 12M HNO₃ and 36M H₂SO₄ (70° C., 2 h); (b) multiple rinsing with bi-distilled H₂O until neutrality.

It is another object of the present invention to provide the method as defined above, wherein said steps of (a) carboxylative opening oxidation-sensitive end-caps, namely, polyCOOH end cluster; and, (b) introducing defect carboxylic (COOH) groups on sidewall surfaces of said oxidized COOH-MWCNTs; are performed simultaneously.

It is another object of the present invention to provide the method as defined above, wherein said step of COOH activating the polyCOOH shell is performed by steps of (a) admixing aqueous N′-(3-dimethylaminopropyl)-N-ethyl-carbodiimide (EDC); (b) covalently attaching at least one selected from a group consisting of Thp-containing linker thiophene-3-ethanol, hydroxylated or aminated polypyrrole/carbazolyl (Pyr/Cbz)-containing linkers; Pyr/Cbz/Th bulk monomers; Pyr/Cbz/Th linkers and any combination thereof.

It is another object of the present invention to provide the method as defined above, wherein said step of COOH activating the polyCOOH shell is performed by using about 3.0 mg or about 15 mmoles of EDC.

It is another object of the present invention to provide the method as defined above, wherein said step of COOH activating the polyCOOH shell is performed by using about 1.0 mL H₂O.

It is another object of the present invention to provide the method as defined above, wherein said step of COOH activating the polyCOOH shell is performed for about 1 h.

It is another object of the present invention to provide the method as defined above, wherein said step of COOH activating the polyCOOH shell is performed at room temperature.

It is another object of the present invention to provide the method as defined above, wherein said step of covalently attaching at least one selected from a group consisting of Thp-containing linker, thiophene-3-ethanol, hydroxylated or aminated polypyrrole/carbazolyl (Pyr/Cbz)-containing linkers; Pyr/Cbz/Th bulk monomers; Pyr/Cbz/Th linkers and any combination thereof is performed by adding said linker 1.0 equiv./EDC in about 1.0 mL CH₃CN.

It is another object of the present invention to provide the method as defined above, wherein said step of covalently attaching is performed for about 10 hours.

It is another object of the present invention to provide the method as defined above, wherein said step of covalently attaching is performed at about room temperature.

It is another object of the present invention to provide the method as defined above, wherein said EDC reacts with MWCNT carboxylic acid groups to form an active O-acylisourea intermediate.

It is another object of the present invention to provide the method as defined above, wherein said intermediate can be easily displaced by nucleophilic attack using the corresponding hydroxylated Th-containing linker.

It is another object of the present invention to provide the method as defined above, wherein said step of COOH activating the polyCOOH shell is performed by using at least one selected from a group consisting of PEG-passivated oxidized MWCNTs, polyvinylpyrrolidone (PVP), polycarbonates (PCs), polyesters (PEs), polysiloxanes; and any combination thereof.

It is another object of the present invention to provide the method as defined above, wherein said PEG is α,ω-bis-methoxy PEG_{2,000} polymer.

It is another object of the present invention to provide the method as defined above, wherein said PEG is α,ω-bis-methoxy PEG polymer.

It is another object of the present invention to provide the method as defined above,

wherein the molecular weight of said PEG is MW=2,000 Daltons.

It is another object of the present invention to provide the method as defined above, wherein about 30.0 mL to about 3.0 mL of distilled water of said PEG is used.

It is another object of the present invention to provide the method as defined above, wherein said step of PEG-passivated oxidized MWCNTs is performed for about 20 min incubation.

It is another object of the present invention to provide the method as defined above, wherein said step of PEG-passivated oxidized MWCNTs is performed at about 20° C.

It is another object of the present invention to provide the method as defined above, wherein said step of Liquid Phase Polymerization (LPP) oxidative deposing said polyCOOH polyTh-CP polymers is performed by at least one selected from a group consisting of (a) Th-containing MWCNT; (b) acidic Th-based LPP monomer thiophene-3-yl acetic acid.

It is another object of the present invention to provide the method as defined above, wherein said step of Liquid Phase Polymerization (LPP) oxidative deposing said polymers is performed by at least one selected from a group consisting of (i) former type of “nucleophilized” Th-containing MWCNTs; (ii) acidic Th-based LPP monomer thiophene-3-yl acetic acid and; (iii) Pyr/polyPyr; (iv) Pyr/polyPyr; any combination thereof.

It is another object of the present invention to provide the method as defined above, wherein said step of Liquid Phase Polymerization (LPP) oxidative deposing said polymers is performed while using cationic cetyltrimethylammonium bromide (CTAB) concentration in the rang of about 0.01 to about 0.1 M for at least 1 hour.

It is another object of the present invention to provide the method as defined above, wherein said selectively deposition is performed in a Liquid Phase Polymerization conditions (LPP conditions) selected from a group consisting of (a) concentration of cationic cetyltrimethylammonium bromide surfactant (CTAB) in the range of about 0[M] to about 0.1M; (b) the amount of Thiophene-3-yl acetic acid is in the range of 10.0 mg to about 35 mg; (c) the amount of Thiophene-3-yl acetic acid is in the range of 0.01 mmol to about 0.2 mmol; (d) the amount of Oxidant is in the range of 1.0 equiv./Th-monomer to about 3.5 equiv./Th-monomer; (e) the amount of Monomer solvent is in the range of 1.0 mL to about 3.5 mL; (f) Temp. of polymerization is in the range of 0 degrees to about 10 degree; (g) Time of polymerization is in the range of 0.5 hours to about 2 hours; and any combination thereof.

It is another object of the present invention to provide the method as defined above, wherein said Oxidant is Anhydrous FeCl₃.

It is another object of the present invention to provide the method as defined above, wherein said Monomer solvent is Distilled CHCl₃.

It is another object of the present invention to provide a “growth from surface” method for fabricating functional dual phase Conducting Polymer/Polythiophene (CP/PolyTh)-Carbon Nanotube (CNT), comprising:
a. obtaining Multi-walled Carbon Nanotubes (MWCNT);
b. oxidized said MWCNTs to obtain oxidized COOH-MWCNTs; thereby (a) carboxylative opening oxidation-sensitive end-caps (polyCOOH end cluster); and, (b) introducing defect carboxylic (COOH) groups onto predetermined areas of said oxidized COOH-MWCNTs;
c. COOH activating the polyCOOH shell (namely, the surface) using various COOH activating species;
d. Liquid Phase Polymerization (LPP) oxidative deposing polymers selected from said polyCOOH polyTh-CP polymers, polyCOOH polyTh-, polyEDOT (PEDOT)-, polyTh polyCOOH poly(thiophenyl-3 acetic acid, thiophenyl-3 acetic acid/EDOT, polyX, wherein X is elected from COOH, OH, NH₂, polyCbz/polyPyr CP polymers and related combinatorial mixtures, polyCOOH PEDOT-poly(thiophenyl-3 acetic acid); thereby providing said dual phase Conducting Polymer/Polythiophene (CP/PolyTh)-Carbon Nanotube (CNT).

It is another object of the present invention to provide the method as defined above, wherein said predetermined area are selected from a group consisting of sidewall surfaces of said oxidized COOH-MWCNTs or CNT extremities or topologically selectively at only oxidized extremities of pegylated oxidized polyTh-decorated MWCNTs, end-decorated, selectively end-decorated Th-CNTs and any combination thereof.

It is another object of the present invention to provide the method as defined above, wherein said step of obtaining said Multi-walled Carbon Nanotubes (MWCNT) is obtained by chemical vapor deposition (CVD) and possess average diameters/lengths of 140±30 nm/7±2 nm respectively.

It is another object of the present invention to provide the method as defined above, wherein said MWCNT are composed of 340-530 graphitic layers and disclose purity higher than 90% as determined by thermogravimetric analysis (TGA).

It is another object of the present invention to provide the method as defined above, wherein said step of oxidizing said MWCNTs is performed by known wet-chemistry protocol.

It is another object of the present invention to provide the method as defined above, wherein said step of oxidizing said MWCNT by known wet-chemistry protocol is performed by steps of (a) oxidative acidic 1/1 v/v mixture of concentrated 12M HNO₃ and 36M H₂SO₄ (70° C., 2 h); (b) multiple rinsing with bi-distilled H₂O until neutrality.

It is another object of the present invention to provide the method as defined above, wherein said steps of (a) carboxylative opening oxidation-sensitive end-caps (polyCOOH end cluster); and, (b) introducing defect carboxylic (COOH) groups on sidewall surfaces of said oxidized COOH-MWCNTs; are performed simultaneously.

It is another object of the present invention to provide the method as defined above, wherein said step of COOH activating the polyCOOH shell is performed by steps of (a) admixing aqueous N′-(3-dimethylaminopropyl)-N-ethyl-carbodiimide (EDC); (b) covalently attaching at least one selected from a group consisting of Thp-containing linker thiophene-3-ethanol, hydroxylated or aminated polypyrrole/carbazolyl (Pyr/Cbz)-containing linkers; Pyr/Cbz/Th bulk monomers; Pyr/Cbz/Th linkers and any combination thereof.

It is another object of the present invention to provide the method as defined above, wherein said step of COOH activating (polyCOOH shell) is performed by using about 3.0 mg (or about 15.7 mmoles) of EDC.

It is another object of the present invention to provide the method as defined above, wherein said step of COOH activating the polyCOOH shell is performed for about 1 h.

It is another object of the present invention to provide the method as defined above, wherein said step of COOH activating the polyCOOH shell is performed at room temperature.

It is another object of the present invention to provide the method as defined above, wherein said step of covalently attaching at least one selected from a group consisting of Thp-containing linker, thiophene-3-ethanol, hydroxylated or aminated polypyrrole/carbazolyl (Pyr/Cbz)-containing linkers; Pyr/Cbz/Th bulk monomers; Pyr/Cbz/Th linkers and any combination thereof is performed by adding said linker 1.0 equiv./EDC in about 1.0 mL CH₃CN.

It is another object of the present invention to provide the method as defined above, wherein said step of covalently attaching is performed for about 10 hours.

It is another object of the present invention to provide the method as defined above, wherein said step of covalently attaching is performed at about room temperature.

It is another object of the present invention to provide the method as defined above, wherein said EDC reacts with MWCNT carboxylic acid groups to form an active O-acylisourea intermediate.

It is another object of the present invention to provide the method as defined above, wherein said intermediate can be easily displaced by nucleophilic attack using the corresponding hydroxylated Th-containing linker.

It is another object of the present invention to provide the method as defined above, wherein said step of COOH activating the polyCOOH shell is performed by using at least one selected from a group consisting of PEG-passivated oxidized MWCNTs, polyvinylpyrrolidone (PVP), polycarbonates (PCs), polyesters (PEs), polysiloxanes; and any combination thereof.

It is another object of the present invention to provide the method as defined above, wherein said PEG is α,ω-bis-methoxy PEG polymer.

It is another object of the present invention to provide the method as defined above,

wherein the molecular weight of said PEG is MW=2,000 Daltons.

It is another object of the present invention to provide the method as defined above, wherein about 30.0 mL to about 3.0 mL of distilled water of said PEG is used.

It is another object of the present invention to provide the method as defined above, wherein said step of PEG-passivated oxidized MWCNTs is performed for about 20 min incubation.

It is another object of the present invention to provide the method as defined above, wherein said step of PEG-passivated oxidized MWCNTs is performed at about 20° C. It is another object of the present invention to provide the method as defined above, wherein said step of Liquid Phase Polymerization (LPP) oxidative deposing said polyCOOH polyTh-CP polymers is performed by at least one selected from a group consisting of (a) Th-containing MWCNTs; (b) acidic Th-based LPP monomer thiophene-3-yl acetic acid.

It is another object of the present invention to provide the method as defined above, wherein said step of Liquid Phase Polymerization (LPP) oxidative deposing said polymers is performed by at least one selected from a group consisting of (i) former type of “nucleophilized” Th-containing MWCNTs; (ii) acidic Th-based LPP monomer thiophene-3-yl acetic acid; (iii) Pyr/polyPyr; (iv) Pyr/polyPyr; and any combination thereof.

It is another object of the present invention to provide the method as defined above, wherein said step of Liquid Phase Polymerization (LPP) oxidative deposing said polymers is performed while using cationic cetyltrimethylammonium bromide (CTAB) concentration in the rang of about 0.01 to about 0.1 M for at least 1 hour.

It is another object of the present invention to provide the method as defined above, additionally comprising step of selectively depositing oxidative Liquid Phase Polymerizations (LPPs) onto the carbon nanotubes (CNTs) surface is performed in a Liquid Phase Polymerization conditions (LPP conditions) selected from a group consisting of (a) concentration of cationic cetyltrimethylammonium bromide surfactant (CTAB) in the range of about 0[M] to about 0.1M; (b) the amount of Thiophene-3-yl acetic acid is in the range of 10.0 mg to about 35 mg; (c) the amount of Thiophene-3-yl acetic acid is in the range of 0.01 mmol to about 0.2 mmol; (d) the amount of Oxidant is in the range of 1.0 equiv./Th-monomer to about 3.5 equiv./Th-monomer; (e) the amount of Monomer solvent is in the range of 1.0 mL to about 3.5 mL; (f) Temp. of polymerization is in the range of 0 degrees to about 10 degree; (g) Time of polymerization is in the range of 0.5 hours to about 2 hours; and any combination thereof.

It is another object of the present invention to provide the method as defined above, wherein said Oxidant is anhydrous FeCl₃.

It is another object of the present invention to provide the method as defined above, wherein said Monomer solvent is Distilled CHCl₃

It is another object of the present invention to provide Th-decorated oxidized MWCNTs for use as nucleophilic nanosized phases in Liquid Phase Polymerization. It is another object of the present invention to provide the Th-decorated oxidized MWCNTs as defined above, wherein said nucleophilic nanosized phases in Liquid Phase Polymerization is provided by the use of Thiophene (Th)-acetic acid precursor for polyCOOH polyTh-CP polymer deposition and covalent attachment. It is another object of the present invention to provide the Th-decorated oxidized MWCNTs as defined above, wherein said decorative oxidized MWCNTs is provided in predetermined locations selected from sidewall, end-decorated, selectively end-decorated Th-CNTs and any combination thereof.

It is another object of the present invention to provide the Th-decorated oxidized MWCNTs as defined above, adapted to provide polymeric CP-chains grown oxidatively in bulk media of oxidative Liquid Phase Polymerizations (LPPs).

It is still an object of the present invention to provide the Th-decorated oxidized MWCNTs as defined above, adapted to provide a selective deposition onto at least one selected from a group consisting of the CNT surface, the CNT sidewall, or at oxidized extremities.

It is lastly an object of the present invention to provide the Th-decorated oxidized MWCNTs as defined above, wherein said selective deposition is provided at controlled amount and surface coverage.

### EXAMPLES

Examples are given in order to prove the embodiments claimed in the present invention. The example, which is a clinical test, describes the manner and process of the present invention and set forth the best mode contemplated by the inventors for carrying out the invention, but are not to be construed as limiting the invention.

### Example 1

### Specific Reagents & Pyr/Cbz-Containing Linkers/LPP Monomers

The MWCNTs used in this study are commercially available from MER Corporation Ltd. (USA). They were produced by chemical vapor deposition (CVD) and possess average diameters/lengths of 140±30 nm/7±2 nm respectively. They are composed of 340-530 graphitic layers and disclose purity higher than 90% as determined by thermogravimetric analysis (TGA). Pyr/Cbz-based LPP monomers 2a:

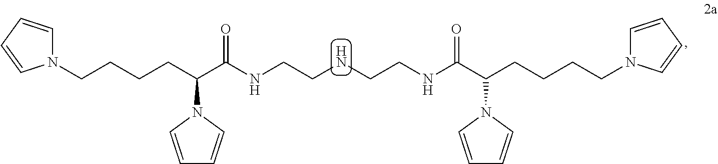

4a:

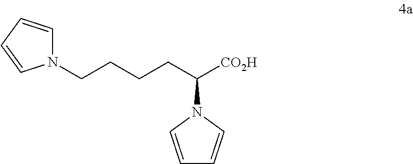 [SMILES: O=C(O)[C@H](CCCCn1cccc1)n1cccc1]

and 2b:

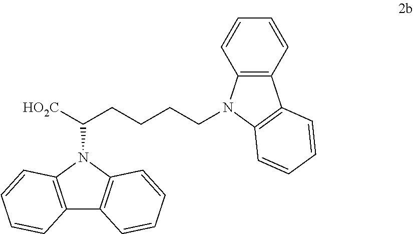

are known compounds.

### Pyr-linkers 1a:

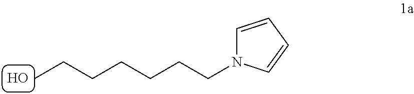

and 3a:

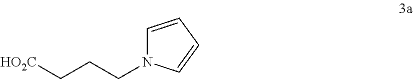 [SMILES: O=C(O)CCCn1cccc1]

have been prepared from the corresponding amino-alcohol/aminoacid 6-amino-hexan-1-ol/4-amino-butanoic acid respectively using a modified Clauson-Kaas reaction (2,5-dimethoxy-tetrahydrofuran, AcOH/1,4-dioxane, 120° C., 1 h and overnight at 25° C.).

The hydroxymethylated Cbz-linker 1b:

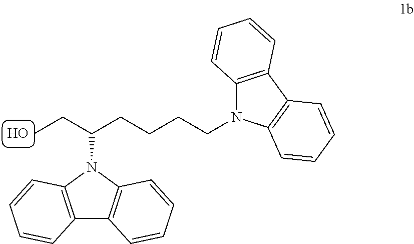

has been quantitatively obtained from the methyl ester of acidic Cbzmonomer 2b:

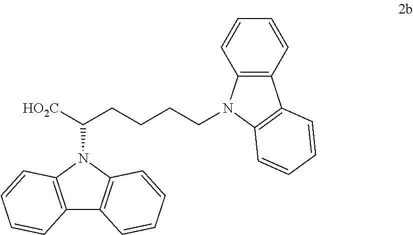

(MeOH, catalytic H2SO4, reflux, 1 h, 95% yield) using a diisobutylaluminium hydride (DIBAL)-mediated reduction (DIBAL, CH2Cl2, 25° C., 2 h).

### Example 2

### Oxidized polyCOOH MWCNTs (c-MWCNTs)

c-MWCNTs were prepared according to a known oxidative wet-chemistry method, i.e. the use of an oxidative acidic 1/1 v/v mixture of concentrated

12M HNO3 and 36M H2SO4 (70° C., 2 h) followed by multiple rinsing with bi-distilled H2O until neutrality. It resulted in the carboxylative opening of oxidation-sensitive end-caps, and in the introduction of defect carboxylic (COOH) groups on sidewall surfaces of oxidized MWCNTs (c-MWCNTs).

### Example 3

### Preparation of “Nucleophilic” d-MWCNTs—Covalent Coupling/Grafting of c-MWCNTs with Pyr/Cbz-Containing Linkers 1a

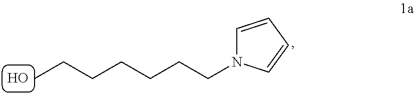

1b:

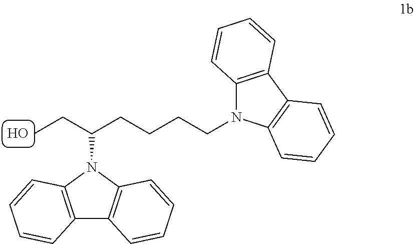

and
2a:

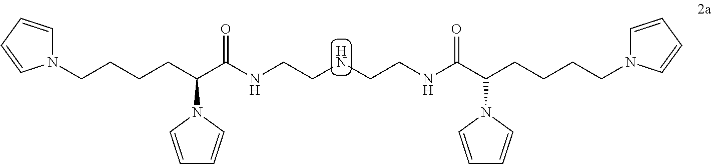

The coupling/grafting chemistry used for the fabrication of intermediate “nucleophilic” d-MWCNTs1-2a,1b made use of an aqueous N′-(3-dimethylaminopropyl)-N-ethyl-carbodiimide (EDC)-mediated activation of the carboxylate functions present on oxidized c-MWCNTs followed by the covalent attachment of Pyr/Cbz-containing linkers 1-2a and 1b [1.0 mg c-MWCNTs, 3.0 mg (15.7 mmoles EDC, 1.0 mL H2O, 1 h, rt), 1-2a and 1b monomers: 1.0 equiv./equiv. EDC dissolved in 1.0 mL CH₃CN, overnight, rt]. Depending on linker structures, obtained d-MWCNTs1-2a,1b contained ester (linkers 1a/1b) or amide bonds (linker 2a). These intermediate “nucleophilic” nanomaterials have been characterized by FT-IR, SEM/TEM, and XPS in order to check for successful linker attachment. In particular, TEM and SEM analyses showed that resulting d-MWCNTs1-2a,1b morphologies (lengths and averaged outer diameters) exactly resembled observed morphologies of starting oxidized c-MWCNTs (results not shown). In addition, FT-IR spectroscopy and surface-sensitive XPS readily confirmed the presence of linked functional groups/aromatic heterocycles following

carboxylate modification by Pyr/Cbz-linkers (FT-IR: SOH stretching peaks at 3458.0 cm-1 (COOH function, strong), δCsp3 peaks at 2853.9 and 2925.1 cm-1, □νCsp2-Csp2 peaks at 1604-1610 and 1454-1600 cm⁻¹ (Pyr and Cbz heterocycles respectively, strong); XPS: presence of the characteristic compositional N is peak (BE=399.55 eV).

### Example 4

### Oxidative Liquid Phase Polymerizations of Pyr/Cbz-Containing LPP Monomers 3-4a and 2b in the Presence of “Nucleophilic” d-MWCNTs1-2a,1b (Typical Procedure)

Each different d-MWCNTs1-2a,1b (25.0 mg) was separately suspended in doubly distilled neutral H2O (8 mL for 1a, 4 mL for 2a & 1b) in the presence of a cationic surfactant cetyltrimethylammonium bromide (CTAB, 364.5 mg & 182.5 mg, 0.1M final concentration). A 1 h-long ultrasonication using a Bransonic [symbol] bath sonicator (42 KHz at full power) afforded well-dispersed aqueous suspensions of each corresponding CTAB/d-MWCNTs1-2a,1b composite material. Then, the related CTAB/d-MWCNTs1a, CTAB/d-WCNTs2a, and CTAB/d-MWCNTs1b-based dispersions were respectively added in this order with the following couples of LPP Pyr/Cbz-monomer/oxidant reagents (magnetic agitation): 3a (80.0 mg, 0.52 mmol)/anhydrous FeCl3 (85.0 mg, 0.52 mmol), 4a (60.0 mg, 0.243 mmol)/FeCl3.6H2O (66.0 mg, 0.243 mmol), and 2b (20.0 mg, 0.05 mmol)/ammonium persulfate [(NH4)2S2O8, APS, 25.0 mg, 1.25 mmol]. LPP monomers 3-4a and 2b were previously dissolved in AcCN (3a, 2.0 mL) and MeCOMe (4a & 2b, 1.0 mL) Both FeCl3.6H2O and APS LPP oxidants were added as neat powders. At LPP completion (4 h, rt), obtained poly(3a)/d-MWCNTs1a, poly(4a)/d-MWCNTs2a, and poly(2b)/d-MWCNTs1b composite materials were then washed in a 1/1 v/v mixture of doubly distilled neutral H2O-monomer solvent mixture (5×10 mL) and decanted by ultra-centrifugation (10,000 rpm, 5×3 min, 100 C). All the resulting purified composites were dried under vacuum (3 h, 10-3 mm Hg, rt) before characterization.

1-65. (canceled)

66. A “growth from the surface” method for selectively depositing oxidative Liquid Phase Polymerizations (LPPs) onto the carbon nanotube (CNT) surface, said method comprising steps of:
a. obtaining Multi-walled Carbon Nanotubes (MWCNT);
b. oxidized said MWCNTs to obtain oxidized COOH-MWCNTs; thereby (a) carboxylative opening oxidation-sensitive end-caps (polyCOOH end cluster); and, (b) introducing defect carboxylic (COOH) groups onto predetermined areas of said oxidized COOH-MWCNTs;
c. COOH activating the polyCOOH shell using various COOH activating species; and,
d. executing Liquid Phase Polymerization (LPP) oxidative depositing polymers selected from said polyCOOH polyTh-CP polymers, polyCOOH polyTh-, polyEDOT (PEDOT)-, polyTh polyCOOH poly(thiophenyl-3 acetic acid, thiophenyl-3 acetic acid/EDOT, polyX, wherein X is elected from COOH, OH, NH₂, polyCbz/polyPyr CP polymers and related combinatorial mixtures, polyCOOH PEDOT-poly(thiophenyl-3 acetic acid)′ thereby selectively depositing said oxidative LPPs onto said CNT surface.

67. The method according to claim 66, wherein at least one of the following is being held true (a) said selectively deposition is performed in a controlled manner and for controlled polymer deposited amounts; (b) said predetermined area are selected from a group consisting of sidewall surfaces of said oxidized COOH-MWCNTs or CNT extremities or topologically selectively at only oxidized extremities of pegylated oxidized polyTh-decorated MWCNTs, end-decorated, selectively end-decorated Th-CNTs and any combination thereof; and any combination thereof.

68. The method according to claim 66, wherein at least one of the following is being held true (a) said step of obtaining Multi-walled Carbon Nanotubes (MWCNT) is performed by chemical vapor deposition (CVD) and possess average diameters/lengths of 140±30 nm/7±2 nm respectively; (b) said MWCNT are composed of about 340 to about 530 graphitic layers and disclose purity higher than 90% as determined by thermogravimetric analysis (TGA); and any combination thereof.

69. The method according to claim 66, wherein said step of oxidizing said MWCNT by conventional wet-chemistry protocol is performed by steps of (a) oxidative acidic 1/1 v/v mixture of concentrated 12M HNO₃ and 36M H₂SO₄, at a temperature of about 70° C. for about 2 hours; (b) multiple rinsing with bi-distilled H₂O until neutrality.

70. The method according to claim 66, wherein said steps of (a) carboxylative opening oxidation-sensitive end-caps, namely, polyCOOH end cluster; and, (b) introducing defect carboxylic (COOH) groups on sidewall surfaces of said oxidized COOH-MWCNTs; are performed simultaneously.

71. The method according to claim 66, wherein said step of COOH activating the polyCOOH shell is performed by steps of (a) admixing aqueous N′-(3-dimethylaminopropyl)-N-ethyl-carbodiimide (EDC); (b) covalently attaching at least one selected from a group consisting of Thp-containing linker, thiophene-3-ethanol, hydroxylated or aminated polypyrrole/carbazolyl (Pyr/Cbz)-containing linkers; Pyr/Cbz/Th bulk monomers; Pyr/Cbz/Th linkers and any combination thereof.

72. The method according to claim 66, wherein said step of COOH activating the polyCOOH shell is performed by using at least one selected from a group consisting of (a) using about 3.0 mg or about 15 mmoles of EDC; (b) said step of COOH activating the polyCOOH shell is performed for about 1 h; (c) said step of COOH activating the polyCOOH shell is performed at room temperature; and any combination thereof.

73. The method according to claim 66, wherein said step of covalently attaching at least one selected from a group consisting of Thp-containing linker, thiophene-3-ethanol, hydroxylated or aminated polypyrrole/carbazolyl (Pyr/Cbz)-containing linkers; Pyr/Cbz/Th bulk monomers; Pyr/Cbz/Th linkers and any combination thereof is performed by adding said linker in about 1.0 equiv./EDC in about 1.0 mL CH₃CN; further wherein said step of covalently attaching is performed for about 10 hours; further wherein said step of covalently attaching is performed at about room temperature.

74. The method according to claim 73, wherein said EDC reacts with MWCNT carboxylic acid groups to form an active O-acylisourea intermediate; further wherein said intermediate can be easily displaced by nucleophilic attack using the corresponding hydroxylated Th-containing linker.

75. The method according to claim 66, wherein said step of COOH activating the polyCOOH shell is performed by using at least one selected from a group consisting of PEG-passivated oxidized MWCNTs, polyvinylpyrrolidone (PVP), polycarbonates (PCs), polyesters (PEs), polysiloxanes; and any combination thereof; further wherein said PEG is α,ω-bis-methoxy PEG polymer; further wherein the molecular weight of said PEG is MW=2,000 Daltons.

76. The method according to claim 75, wherein about 30.0 mL to about 3.0 mL of distilled water of said PEG is used; further wherein said step of PEG-passivated oxidized MWCNTs is performed for about 20 min incubation at about 20° C.

77. The method according to claim 66, wherein said step of Liquid Phase Polymerization (LPP) oxidative deposing said polyCOOH polyTh-CP polymers is performed by at least one selected from a group consisting of (a) Th-containing MWCNT; (b) acidic Th-based LPP monomer thiophene-3-yl acetic acid.

78. The method according to claim 77, wherein at least one of the following is being held true (a) said step of Liquid Phase Polymerization (LPP) oxidative deposing said polymers is performed by at least one selected from a group consisting of (i) former type of “nucleophilized” Th-containing MWCNTs; (ii) acidic Th-based LPP monomer thiophene-3-yl acetic acid; (iii) Pyr/polyPyr; (iv) Pyr/polyPyr; and any combination thereof; (b) said step of Liquid Phase Polymerization (LPP) oxidative deposing said polymers is performed while using cationic cetyltrimethylammonium bromide (CTAB) concentration in the rang of about 0.01 to about 0.1 M for at least 1 hour; and any combination thereof.

79. The method according to claim 66, wherein said selectively deposition is performed in a Liquid Phase Polymerization conditions (LPP conditions) selected from a group consisting of (a) concentration of cationic cetyltrimethylammonium bromide surfactant (CTAB) in the range of about 0[M] to about 0.1M; (b) the amount of Thiophene-3-yl acetic acid is in the range of 10.0 mg to about 35 mg; (c) the amount of Thiophene-3-yl acetic acid is in the range of 0.01 mmol to about 0.2 mmol; (d) the amount of Oxidant is in the range of 1.0 equiv./Th-monomer to about 3.5 equiv./Th-monomer; (e) the amount of Monomer solvent is in the range of 1.0 mL to about 3.5 mL; (f) Temp. of polymerization is in the range of 0 degrees to about 10 degree; (g) Time of polymerization is in the range of 0.5 hours to about 2 hours; and any combination thereof.

80. The method according to claim 79, wherein at least one of the following is being held true (a) said Oxidant is Anhydrous FeCl₃; (b) said Monomer solvent is Distilled CHCl₃ and any combination thereof.

81. A “growth from surface” method for fabricating functional dual phase Conducting Polymer/Polythiophene (CP/PolyTh)-Carbon Nanotube (CNT), comprising:
a. obtaining Multi-walled Carbon Nanotubes (MWCNT);
b. oxidized said MWCNTs to obtain oxidized COOH-MWCNTs; thereby (a) carboxylative opening oxidation-sensitive end-caps (polyCOOH end cluster); and, (b) introducing defect carboxylic (COOH) groups onto predetermined areas of said oxidized COOH-MWCNTs;
c. COOH activating the polyCOOH shell using various COOH activating species;
d. executing Liquid Phase Polymerization (LPP) oxidative depositing polymers selected from said polyCOOH polyTh-CP polymers, polyCOOH polyTh-, polyEDOT (PEDOT)-, polyTh polyCOOH poly(thiophenyl-3 acetic acid, thiophenyl-3 acetic acid/EDOT, polyX, wherein X is elected from COOH, OH, NH₂, polyCbz/polyPyr CP polymers and related combinatorial mixtures, polyCOOH PEDOT-poly(thiophenyl-3 acetic acid); thereby providing said dual phase Conducting Polymer/Polythiophene (CP/PolyTh)-Carbon Nanotube (CNT).

82. The method according to claim 81, wherein at least one of the following is being held true (a) said predetermined area are selected from a group consisting of sidewall surfaces of said oxidized COOH-MWCNTs or CNT extremities or topologically selectively at only oxidized extremities of pegylated oxidized polyTh-decorated MWCNTs, end-decorated, selectively end-decorated Th-CNTs and any combination thereof; (b) said step of obtaining Multi-walled Carbon Nanotubes (MWCNT) is obtained by chemical vapor deposition (CVD) and possess average diameters/lengths of 140±30 nm/7±2 nm respectively; and any combination thereof.

83. The method according to claim 81, wherein said MWCNT are composed of 340-530 graphitic layers and disclose purity higher than 90% as determined by thermogravimetric analysis (TGA);

84. The method according to claim 81, wherein said step of oxidizing said MWCNT by conventional wet-chemistry protocol is performed by steps of (a) oxidative acidic 1/1 v/v mixture of concentrated 12M HNO₃ and 36M H₂SO₄ (70° C., 2 h); (b) multiple rinsing with bi-distilled H₂O until neutrality.

85. The method according to claim 81, wherein said steps of (a) carboxylative opening oxidation-sensitive end-caps (polyCOOH end cluster); and, (b) introducing defect carboxylic (COOH) groups on sidewall surfaces of said oxidized COOH-MWCNTs; are performed simultaneously.

86. The method according to claim 81, wherein at least one of the following is being held true (a) said step of COOH activating the polyCOOH shell is performed by steps of (a) admixing aqueous N′-(3-dimethylaminopropyl)-N-ethyl-carbodiimide (EDC); (b) covalently attaching at least one selected from a group consisting of Thp-containing linker, thiophene-3-ethanol, hydroxylated or aminated polypyrrole/carbazolyl (Pyr/Cbz)-containing linkers; Pyr/Cbz/Th bulk monomers; Pyr/Cbz/Th linkers and any combination thereof; (b) said step of COOH activating the polyCOOH shell is performed by using about 3.0 mg, or about 15.7 mmoles of EDC; (b) said step of COOH activating the polyCOOH shell is performed by using about 3.0 mg, or about 15.7 mmoles of EDC; (c) said step of COOH activating the polyCOOH shell is performed for about 1 h; (d) wherein said step of COOH activating the polyCOOH shell is performed at room temperature; and any combination thereof.

87. The method according to claim 86, wherein at least one of the following is being held true (a) said step of covalently attaching at least one selected from a group consisting of Thp-containing linker, thiophene-3-ethanol, hydroxylated or aminated polypyrrole/carbazolyl (Pyr/Cbz)-containing linkers; Pyr/Cbz/Th bulk monomers; Pyr/Cbz/Th linkers and any combination thereof is performed by adding said linker 1.0 equiv./EDC in about 1.0 mL CH₃CN; (b) said step of covalently attaching is performed for about 10 hours; (c) said step of COOH covalently attaching is performed at about room temperature; (d) said EDC reacts with MWCNT carboxylic acid groups to form an active O-acylisourea intermediate; (e) said intermediate can be easily displaced by nucleophilic attack using the corresponding hydroxylated Th-containing linker; and any combination thereof.

88. The method according to claim 81, wherein at least one of the following is being held true (a) said step of COOH activating the polyCOOH shell is performed by using at least one selected from a group consisting of PEG-passivated oxidized MWCNTs, polyvinylpyrrolidone (PVP), polycarbonates (PCs), polyesters (PEs), polysiloxanes; and any combination thereof; (b) said PEG is α,ω-bis-methoxy PEG polymer; (c) the molecular weight of said PEG is MW=2,000 Daltons; (d) about 30.0 mL to about 3.0 mL of distilled water of said PEG is used; (e) said step of PEG-passivated oxidized MWCNTs is performed for about 20 min incubation; (f) said step of PEG-passivated oxidized MWCNTs is performed at about 20° C. and any combination thereof.

89. The method according to claim 81, wherein at least one of the following is being held true (a) said step of Liquid Phase Polymerization (LPP) oxidative deposing said polyCOOH polyTh-CP polymers is performed by at least one selected from a group consisting of (a) Th-containing MWCNT; (b) acidic Th-based LPP monomer thiophene-3-yl acetic acid; (b) said step of Liquid Phase Polymerization (LPP) oxidative deposing said polymers is performed by at least one selected from a group consisting of (i) former type of “nucleophilized” Th-containing MWCNTs; (ii) acidic Th-based LPP monomer thiophene-3-yl acetic acid; (iii) Pyr/polyPyr; (iv) Pyr/polyPyr; and any combination thereof; (c) said step of Liquid Phase Polymerization (LPP) oxidative deposing said polymers is performed while using cationic cetyltrimethylammonium bromide (CTAB) concentration in the rang of about 0.01 to about 0.1 M for at least 1 hour; and any combination thereof.

90. The method according to claim 81, additionally comprising at least one step selected from a group consisting of (a) selectively depositing oxidative Liquid Phase Polymerizations (LPPs) onto the carbon nanotubes (CNT) surface is performed in a Liquid Phase Polymerization conditions (LPP conditions) selected from a group consisting of (a) concentration of cationic cetyltrimethylammonium bromide surfactant (CTAB) in the range of about 0[M] to about 0.1M; (b) the amount of Thiophene-3-yl acetic acid is in the range of 10.0 mg to about 35 mg; (c) the amount of Thiophene-3-yl acetic acid is in the range of 0.01 mmol to about 0.2 mmol; (d) the amount of Oxidant is in the range of 1.0 equiv./Th-monomer to about 3.5 equiv./Th-monomer; (e) the amount of Monomer solvent is in the range of 1.0 mL to about 3.5 mL; (f) Temp. of polymerization is in the range of 0 degrees to about 10 degree; (g) Time of polymerization is in the range of 0.5 hours to about 2 hours; and any combination thereof; (b) selecting said Oxidant to be Anhydrous FeCl₃; (c) selecting said Monomer solvent to be Distilled CHCl₃; and any combination thereof.

91. A Th-decorated oxidized MWCNTs for use as nucleophilic nanosized phases in Liquid Phase Polymerization.

92. The Th-decorated oxidized MWCNTs of claim 91, wherein said nucleophilic nanosized phases in Liquid Phase Polymerization is provided by the use of Thiophene (Th)-acetic acid precursor for polyCOOH polyTh-CP polymer deposition and covalent attachment.

93. The Th-decorated oxidized MWCNTs of claim 91, wherein said decorative oxidized MWCNTs is provided in predetermined locations selected from sidewall, end-decorated, selectively end-decorated Th-CNTs and any combination thereof.

94. The Th-decorated oxidized MWCNTs of claim 91, adapted to provide polymeric CP-chains grown oxidatively in bulk media of oxidative Liquid Phase Polymerizations (LPPs).

95. The Th-decorated oxidized MWCNTs of claim 91, adapted to provide a selective deposition onto at least one selected from a group consisting of the CNT surface, the CNT sidewall, or at oxidized extremities.

96. The Th-decorated oxidized MWCNTs of claim 95, wherein said selective deposition is provided at controlled amount and surface coverage.
